(19) United States
(12) Patent Application Publication
(10) Pub. No.: US 2025/0235164 A1
(43) Pub. Date: Jul. 24, 2025

(54) METHOD OF PROVIDING SUSTAINED ENERGY

(71) Applicant: PepsiCo, Inc., Purchase, NY (US)

(72) Inventors: Melissa ANDERSON, Lakewood Ranch, FL (US); Damian BROWNE, Ridgefield, CT (US); Danielle BARBARO, Riverdale, NJ (US); Kelly BARNES, Algonquin, IL (US)

(73) Assignee: PepsiCo, Inc., Purchase, NY (US)

(21) Appl. No.: 19/033,269

(22) Filed: Jan. 21, 2025

Related U.S. Application Data
(60) Provisional application No. 63/623,647, filed on Jan. 22, 2024.

(51) Int. Cl. A61B 5/00; A23L 2/38; A23L 2/52; G06N 20/00

(52) U.S. Cl. CPC A61B 5/7282; A23L 2/38; A23L 2/52; G06N 20/00

(57) ABSTRACT

The present disclosure relates to a method of providing sustained energy from a beverage comprising one or more stimulants, such as caffeine, comprising (a) administering the beverage comprising one or more stimulants to a consumer; (b) measuring the consumer's plasma concentration of the one or more stimulants over a first time period; (c) measuring the consumer's visual analog scale (VAS) response over a second time period; and (d) increasing or decreasing the amount of the one or more stimulants in the beverage based on the measurements from (b) and (c). The method can further comprise measuring a baseline for each of the consumer's plasma concentration and VAS response prior to (a). The method can also further comprise measuring one or more vital signs, such as heart rate or blood pressure, of the consumer prior to (d) in a third time period.

### BACKGROUND

An energy drink is a type of beverage that contains one or more stimulant compounds, such as caffeine, to provide mental and/or physical stimulation (“energy”). The energy or alertness provided by energy drinks can range from weak energy to a jolt of energy followed by an energy crash (e.g., “rollercoaster energy”). Caffeine consumption has been consistently shown to acutely improve alertness, attention, vigilance, and reaction time but prior research on such effects typically only show benefits between 60 and 120 minutes post-consumption. However, consuming too much of a given stimulant, especially in an attempt to gain sustained energy and alertness, can have adverse effects on consumers, such as jitteriness or increased heart rate.

Thus, there is a need for a method of providing sustained energy from a beverage comprising one or more stimulants.

### BRIEF SUMMARY

The present disclosure relates to a method of providing sustained energy from a beverage comprising one or more stimulants, the method comprising: (a) administering the beverage comprising one or more stimulants to a consumer; (b) measuring the consumer's plasma concentration of the one or more stimulants over a first time period; (c) measuring the consumer's visual analog scale (VAS) response over a second time period; and (d) increasing or decreasing the amount of the one or more stimulants in the beverage based on the measurements from (b) and (c).

The present disclosure further relates to a method of providing sustained energy from a beverage comprising one or more stimulants, the method comprising: (a) administering the beverage comprising an amount of one or more stimulants to a consumer; (b) measuring the consumer's plasma concentration of the amount of one or more stimulants over a first time period; (c) measuring the consumer's visual analog scale (VAS) response over a second time period; (d) generating, using a trained machine learning model, a stimulant amount based on the measurements from (b) and (c); and (e) increasing or decreasing the amount of the one or more stimulants in the beverage to match the stimulant amount.

In some aspects, the one or more stimulants is selected from the group consisting of a methylxanthine, a B vitamin, yerba mate, guarana, ephedra, taurine, carnitine, creatine, sucralose, maltodextrin, acai, glucuronolactone, yohimbine, ginseng, gingko biloba, kola nut, alpinia galanga, L-theanine, mango leaf, ornithine, blackcurrant, ashwagandha, coffeeberry, arginine silicate, curcumin, methylliberine, sideritis scardica, green oat extract, hawthorn, holy basil, lemon balm, Rhodiola rosea, rosemary, sage, spearmint, theacrine, tyrosine, cocoa flavanols, CoQ10, moringa, cacao, guayusa, and any combination thereof. In some aspects, the methylxanthine is caffeine, theobromine, theophylline, or any combination thereof. In some aspects, the B vitamin is thiamine, riboflavin, niacin, pantothenic acid, pyridoxine, biotin, folate, cobalamin, cyanocobalamin, inositol, or any combination thereof. In some aspects, the one or more stimulants comprises caffeine.

In some aspects, the beverage further comprises water and at least one additive selected from a sweetener, a preservative, color, a flavor, an antioxidant, a nutrient, and any combination thereof. In some aspects, the beverage further comprises water and at least one nutrient.

In some aspects, the first time period is about 30 minutes to about 15 hours after the beverage has been administered. In some aspects, the first time period is about 30 minutes to 12 hours after the beverage has been administered.

In some aspects, the consumer's plasma concentration is measured about every 30 minutes during the first time period.

In some aspects, the second time period is about 30 minutes to about 15 hours after the beverage has been administered. In some aspects, the second time period is about 30 minutes to 12 hours after the beverage has been administered.

In some aspects, the consumer's VAS response is measured about every 60 minutes during the second time period.

In some aspects, the consumer's VAS response comprises measuring one or more factors selected from alertness, headache, irritability, jitteriness, lightheadedness, overall mood, relaxation, tension, tiredness, or any combination thereof.

In some aspects, the method further comprises measuring a baseline for each of the consumer's plasma concentration and VAS response prior to (a). In some aspects, the baseline is measured at about 5 to 60 minutes prior to (a). In some aspects, the baseline is measured at about 5 to 30 minutes prior to (a).

In some aspects, the method further comprises measuring one or more vital signs of the consumer prior to (d) in a third time period. In some aspects, the one or more vital signs comprises heart rate, systolic blood pressure, diastolic blood pressure, or any combination thereof. In some aspects, the consumer's one or more vital signs is measured about every 120 minutes during the third time period.

In some aspects, the amount of the one or more stimulants in the beverage are increased or decreased to provide a beverage with sustained energy for about 2 hours or more (e.g., about 5 hours or more).

In some aspects, the amount of the one or more stimulants in the beverage is increased in the beverage based on the measurements from (b) and (c).

In some aspects, the amount of the one or more stimulants in the beverage is decreased in the beverage based on the measurements from (b) and (c).

In some aspects, the amount of the one or more stimulants in the beverage is increased based on a desired VAS response. In some aspects, the amount of the one or more stimulants in the beverage is decreased based on a desired VAS response.

In some aspects, the amount of the one or more stimulants in the beverage is increased based on a desired vital sign measurement. In some aspects, the amount of the one or more stimulants in the beverage is decreased based on a desired vital sign measurement.

In some aspects, the amount of the one or more stimulants in the beverage is increased based on consumer preference data. In some aspects, the amount of the one or more stimulants in the beverage is decreased based on consumer preference data.

In some aspects, the machine learning model is used to determine the predicted optimal amount of stimulant based on a desired VAS response. In some aspects, the machine learning model is used to determine the predicted optimal combination of stimulants based on a desired VAS response.

In some aspects, the machine learning model is used to determine a predicted optimal amount of stimulant based on a desired vital sign measurement. In some aspects, the machine learning model is used to determine the optimal combination of stimulants based on a desired vital sign measurement.

In some aspects, the machine learning model is used to determine a predicted optimal amount of stimulant based on consumer preference data. In some aspects, the machine learning model is used to determine the predicted optimal combination of stimulants based on consumer preference data.

In some aspects, the machine learning model is used to determine the predicted optimal value for the first time period or the second time period.

In some aspects, the present disclosure relates to a method of providing sustained energy from a beverage comprising one or more stimulants, the method comprising: (a) providing user input to a formulation machine learning model, wherein the formulation machine learning is trained to generate an optimized formulation based on the user input, and wherein the user input comprises a desired VAS response; (b) outputting, by the formulation machine learning model, the optimized formulation, wherein the optimized formulation comprises a combination of the one or more stimulants including at least one of an amount of the one or more stimulants and a type of the one or more stimulants; and (c) providing the optimized formulation to a beverage mixer for producing the beverage based on the combination of the one or more stimulants.

In some aspects of this method, the one or more stimulants is selected from the group consisting of a methylxanthine, a B vitamin, yerba mate, guarana, ephedra, taurine, carnitine, creatine, sucralose, maltodextrin, acai, glucuronolactone, yohimbine, ginseng, gingko biloba, kola nut, alpinia galanga, L-theanine, mango leaf, ornithine, blackcurrant, ashwagandha, coffeeberry, arginine silicate, curcumin, methylliberine, sideritis scardica, green oat extract, hawthorn, holy basil, lemon balm, Rhodiola rosea, rosemary, sage, spearmint, theacrine, tyrosine, cocoa flavanols, CoQ10, moringa, cacao, guayusa, and any combination thereof. In some aspects, the methylxanthine is caffeine, theobromine, theophylline, or any combination thereof. In some aspects, the B vitamin is thiamine, riboflavin, niacin, pantothenic acid, pyridoxine, biotin, folate, cobalamin, cyanocobalamin, inositol, or any combination thereof. In some aspects, the one or more stimulants comprises caffeine.

In some aspects, the beverage further comprises water and at least one additive selected from a sweetener, a preservative, color, a flavor, an antioxidant, a nutrient, and any combination thereof.

In some aspects, the combination of the one or more stimulants comprises one stimulant, wherein the optimized formulation comprises at least one of an optimized amount of the one stimulant and a type of the one stimulant, and where the desired physical response is a desired VAS response.

In some aspects, the combination of one or more stimulants comprises a plurality of stimulants, wherein the optimization formulation comprises at least one of optimized amounts of the plurality of stimulants and types of the plurality of stimulants, and wherein the desired physical response is a desired VAS response. In some aspects, the combination of one or more stimulants comprises one stimulant, wherein the optimized formulation comprises at least one of an optimized amount of the one stimulant and a type of the one stimulant, and wherein the desired physical response is a desired vital sign measurement. In some aspects, the combination of one or more stimulants comprises a plurality of stimulants, wherein the optimization formulation comprises at least one of optimized amounts of the plurality of stimulants and types of the plurality of stimulants, and wherein the desired physical response is a desired vital sign measurement. In some aspects, the combination of one or more stimulants comprises one stimulant, wherein the optimized formulation comprises at least one of an optimized amount of the one stimulant and a type of the one stimulant, and wherein the user input further comprises consumer preference data. In some aspects, combination of one or more stimulants comprises a plurality of stimulants, wherein the optimization formulation comprises at least one of optimized amounts of the plurality of stimulants and types of the plurality of stimulants, and wherein the machine learning model is used to determine the optimal combination of stimulants based on consumer preference data.

Additional embodiments and advantages of the disclosure will be set forth, in part, in the description that follows, and will flow from the description, or can be learned by practice of the disclosure.

It is to be understood that both the foregoing summary and the following detailed description are exemplary and explanatory only, and do not restrict the scope of the claims.

### BRIEF DESCRIPTION OF THE DRAWINGS/FIGURES

FIG. 1 is an example of a visual analog scale (VAS) response consisting of a line (e.g., about a 10 cm line) with verbal anchors at either end. Subjects are instructed to place a vertical line (|) at the point that best reflects the subject's current symptom level.

FIG. 2 is a graph of the least-squares mean alertness scores versus time after dose (hours). Δ with a dashed line is for an encapsulated caffeine (EC) beverage comprising 160 mg caffeine. ▴ is for an immediate-release caffeine (IRC) beverage comprising 160 mg caffeine. ◯ with a dashed line is for an encapsulated caffeine beverage comprising 250 mg caffeine. ● is for an immediate-release caffeine beverage comprising 250 mg caffeine.

FIG. 3 is a graph of the least-squares mean heart rate (beats per minute) versus time after dose (hours). Δ with a dashed line is for an encapsulated caffeine (EC) beverage comprising 160 mg caffeine. ▴ with a solid line is for an immediate-release caffeine beverage comprising 160 mg caffeine. ◯ with a dashed line is for an encapsulated caffeine beverage comprising 250 mg caffeine. ● with a solid line is for an immediate-release caffeine beverage comprising 250 mg caffeine.

FIG. 4 is a graph of the least-squares mean systolic blood pressure (mmHg) versus time after dose (hours). Δ with a dashed line is for an EC beverage comprising 160 mg caffeine. ▴ with a solid line is for an immediate-release caffeine beverage comprising 160 mg caffeine. ◯ with a dashed line is for an EC beverage comprising 250 mg caffeine. ● with a solid line is for an immediate-release caffeine beverage comprising 250 mg caffeine.

FIG. 5 is a graph of the least-squares mean diastolic blood pressure (mmHg) versus time after dose (hours). Δ with a dashed line is for an EC beverage comprising 160 mg caffeine. ▴ with a solid line is for an immediate-release caffeine beverage comprising 160 mg caffeine. ◯ with a dashed line is for an EC beverage comprising 250 mg caffeine. ● with a solid line is for an immediate-release caffeine beverage comprising 250 mg caffeine.

FIG. 6 is a graph of the least-squares mean headache scores versus time after dose (hours). Δ with a dashed line is for an EC beverage comprising 160 mg caffeine. ▴ with a solid line is for an immediate-release caffeine beverage comprising 160 mg caffeine. ◯ with a dashed line is for an EC beverage comprising 250 mg caffeine. ● with a solid line is for an immediate-release caffeine beverage comprising 250 mg caffeine.

FIG. 7 is a graph of the least-squares mean irritable scores versus time after dose (hours). Δ with a dashed line is for an EC beverage comprising 160 mg caffeine. ▴ with a solid line is for an immediate-release caffeine beverage comprising 160 mg caffeine. ◯ with a dashed line is for an EC beverage comprising 250 mg caffeine. ● with a solid line is for an immediate-release caffeine beverage comprising 250 mg caffeine.

FIG. 8 is a graph of the least-squares mean jittery scores versus time after dose (hours). Δ with a dashed line is for an EC beverage comprising 160 mg caffeine. ▴ with a solid line is for an immediate-release caffeine beverage comprising 160 mg caffeine. ◯ with a dashed line is for an EC beverage comprising 250 mg caffeine. ● with a solid line is for an immediate-release caffeine beverage comprising 250 mg caffeine.

FIG. 9 is a graph of the least-squares mean light-headed scores versus time after dose (hours). Δ with a dashed line is for an EC beverage comprising 160 mg caffeine. ▴ with a solid line is for an immediate-release caffeine beverage comprising 160 mg caffeine. ◯ with a dashed line is for an EC beverage comprising 250 mg caffeine. ● with a solid line is for an immediate-release caffeine beverage comprising 250 mg caffeine.

FIG. 10 is a graph of the least-squares mean overall mood scores versus time after dose (hours). Δ with a dashed line is for an EC beverage comprising 160 mg caffeine. ▴ with a solid line is for an immediate-release caffeine beverage comprising 160 mg caffeine. ◯ with a dashed line is for an EC beverage comprising 250 mg caffeine. ● with a solid line is for an immediate-release caffeine beverage comprising 250 mg caffeine.

FIG. 11 is a graph of the least-squares mean pounding heart scores versus time after dose (hours). Δ with a dashed line is for an EC beverage comprising 160 mg caffeine. ▴ with a solid line is for an immediate-release caffeine beverage comprising 160 mg caffeine. ◯ with a dashed line is for an EC beverage comprising 250 mg caffeine. ● with a solid line is for an immediate-release caffeine beverage comprising 250 mg caffeine.

FIG. 12 is a graph of the least-squares mean relaxed scores versus time after dose (hours). Δ with a dashed line is for an EC beverage comprising 160 mg caffeine. ▴ with a solid line is for an immediate-release caffeine beverage comprising 160 mg caffeine. ◯ with a dashed line is for an EC beverage comprising 250 mg caffeine. ● with a solid line is for an immediate-release caffeine beverage comprising 250 mg caffeine.

FIG. 13 is a graph of the least-squares mean tense scores versus time after dose (hours). Δ with a dashed line is for an EC beverage comprising 160 mg caffeine. ▴ with a solid line is for an immediate-release caffeine beverage comprising 160 mg caffeine. ◯ with a dashed line is for an EC beverage comprising 250 mg caffeine. ● with a solid line is for an immediate-release caffeine beverage comprising 250 mg caffeine.

FIG. 14 is a graph of the least-squares mean tired scores versus time after dose (hours). Δ with a dashed line is for an EC beverage comprising 160 mg caffeine. ▴ with a solid line is for an immediate-release caffeine beverage comprising 160 mg caffeine. ◯ with a dashed line is for an EC beverage comprising 250 mg caffeine. ● with a solid line is for an immediate-release caffeine beverage comprising 250 mg caffeine.

FIG. 15 is a plot of both (i) the mean plasma caffeine concentration (ng/mL) versus time of day and (ii) the self-reported alertness (%) versus time of day. □ with a dashed line is for a 160 mg IRC beverage measuring alertness. ⋄ with a dashed line is for a 250 mg IRC beverage measuring alertness. ♦ with a solid line is for a 160 mg IRC beverage measuring mean plasma concentration. ▪ with a solid line is for a 250 mg TRC beverage measuring mean plasma concentration.

FIG. 16 is a plot of self-reported alertness and tiredness (%) versus hours post-consumption of the beverage. ● with a solid line is for a 160 mg TRC beverage measuring alertness. ● with a dashed line is for a 160 mg IRC beverage measuring tiredness.

FIG. 17 is a plot of self-reported alertness and tiredness (%) versus hours post-consumption of the beverage. ▪ with a solid line is for a 250 mg TRC beverage measuring alertness. ▪ with a dashed line is for a 250 mg IRC beverage measuring tiredness.

FIG. 18 is an example formulation optimization system for generating optimal formulations of ingredients for a beverage using a trained machine learning model based on desired VAS responses.

### DETAILED DESCRIPTION

The headings provided herein are not limitations of the various embodiments of the disclosure, which can be defined by reference to the specification as a whole. It is also to be understood that the terminology used herein is for the purpose of describing particular embodiments only, and is not intended to be limiting, since the scope of the present disclosure will be limited only by the appended claims.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

The articles “a,” “an,” and “the” are used herein to refer to one or to more than one (i.e., to at least one) of the grammatical object of the article. By way of example, “an element” means one element or more than one element.

As used herein, the term “about” means within an acceptable error range for the particular value as determined by one of ordinary skill in the art, which depends in part on how the value is measured or determined, i.e., the limitations of the measurement system. For example, “about” can mean a range of up to 20% (e.g., up to 10%, up to 5%, or up to 1%) of a given value.

The term “at least” prior to a number or series of numbers is understood to include the number associated with the term “at least,” and all subsequent numbers or integers that could logically be included, as clear from context. When at least is present before a series of numbers or a range, it is understood that “at least” can modify each of the numbers in the series or range. For example, “at least 3” means at least 3, at least 4, at least 5, etc. When at least is present before a component in a method step, then that component is included in the step, whereas additional components are optional.

As used herein, the terms “comprises,” “comprising,” “having,” “including,” “containing,” and the like are open-ended terms meaning “including, but not limited to.” To the extent a given embodiment disclosed herein “comprises” certain elements, it should be understood that present disclosure also specifically contemplates and discloses embodiments that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an embodiment are included.

As used herein, the terms “consists of,” “consisting of,” and the like are to be construed as closed terms, such that an embodiment “consisting of” a particular set of elements excludes any element, step, or ingredient not specified in the embodiment.

As used herein, the term “measure” and variations thereof can encompass the meaning of a respective term, such as “determine,” “calculate,” and variations thereof.

As used herein, the term “first time period” refers to a set period of time in which the consumer's plasma concentration of one or more stimulants is measured from a drawn blood sample (e.g., a venous blood sample). The first time period can be any suitable amount of time to measure the pharmacokinetics of the stimulant(s). In general, the first time period will begin within minutes (e.g., within about 60 min, within about 50 min, within about 40 min, within about 30 min, within about 20 min, or within about 10 min) after the beverage is administered to the consumer. In general, the first time period will end within hours (e.g., within about 15 hr, within about 12 hr, within about 10 hr, within about 8 hr, within about 6 hr, within about 4 hr, or within about 2 hr) after the beverage is administered to the consumer.

As used herein, the term “intermittently” refers to two steps occurring on an alternating schedule. For example, a first step can be performed every 30 minutes and a second step can be performed every 60 minutes, such that the frequency of the second step is intermittent relative to the frequency of the first step.

As used herein, the term “second time period” refers to a set period of time in which the consumer's visual analog scale (VAS) response is measured. The second time period can be any suitable amount of time to evaluate the consumer's response factors (e.g., alert, tired, headache, jittery, light-headed, headache, etc.). In general, the second time period will begin within minutes (e.g., within about 60 min, within about 50 min, within about 40 min, within about 30 min, within about 20 min, or within about 10 min) after the beverage is administered to the consumer. In general, the first time period will end within hours (e.g., within about 15 hr, within about 12 hr, within about 10 hr, within about 8 hr, within about 6 hr, within about 4 hr, or within about 2 hr) after the beverage is administered to the consumer.

As used herein, the term “simultaneously” refers to two steps occurring at the same time. The term “near simultaneously” refers to two steps occurring as close together as practically possible (e.g., within minutes of each other, such as within about 10 min of each other, within about 5 min of each other, within about 2 min of each other, within about 1 min of each other).

As used herein, the term “stimulant” refers to a small molecule compound that can boost mood, increase one's ability to focus, and/or improve vigor and sociability. The stimulant is generally regarded as safe (GRAS) for use in a beverage.

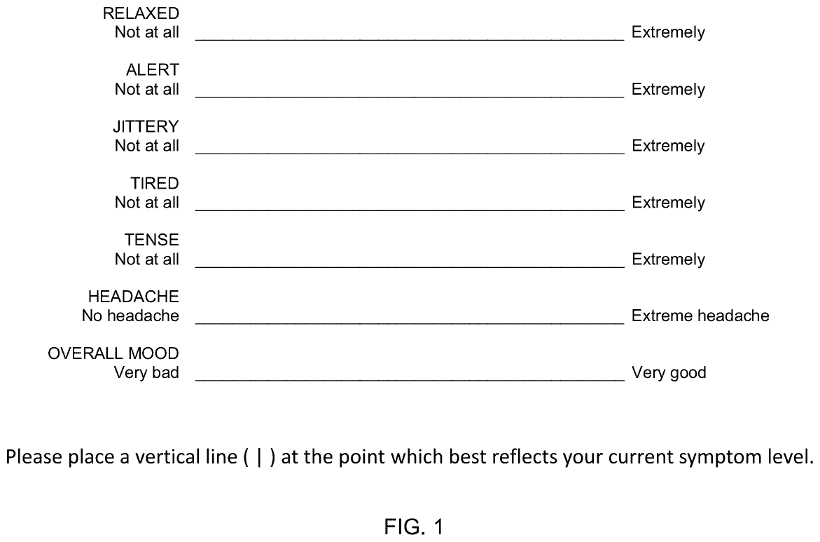

As used herein, the term “visual analog scale response” or “VAS response” is a subjective measure of a subject's (e.g., consumer's) experience. In general, a VAS consists of a line (e.g., about a 10 cm line) with verbal anchors at either end (e.g., “none,” “not at all,” and “very bad” on the far left and “extreme, “extremely,” and “very good” on the far right). See FIG. 1. To provide a response, the subject places a mark at a point on the line corresponding to the subject's rating of each factor at the time of being questioned. The VAS response can comprise measuring one or more factors of a consumer's experience, such as alertness, headache, irritability, jitteriness, lightheadedness, overall mood, relaxation, tension, tiredness, or any combination thereof. Responses can be numerically quantified, and in some cases, several responses can be combined to provide an overall VAS response.

### Methods of the Disclosure

The present disclosure relates to a method of providing sustained energy from a beverage comprising one or more stimulants, the method comprising: (a) administering the beverage comprising one or more stimulants to a consumer; (b) measuring the consumer's plasma concentration of the one or more stimulants over a first time period; (c) measuring the consumer's visual analog scale (VAS) response over a second time period; and (d) increasing or decreasing the amount of the one or more stimulants in the beverage based on the measurements from (b) and (c).

The present disclosure further relates to a method of providing sustained energy from a beverage comprising one or more stimulants, the method comprising: (a) administering the beverage comprising an amount of one or more stimulants to a consumer; (b) measuring the consumer's plasma concentration of the amount of one or more stimulants over a first time period; (c) measuring the consumer's visual analog scale (VAS) response over a second time period; (d) generating, using a trained machine learning model, a stimulant amount based on the measurements from (b) and (c); and (e) increasing or decreasing the amount of the one or more stimulants in the beverage to match the stimulant amount.

The beverage (e.g., an energy drink) can have any suitable composition as long as the beverage composition comprises at least water and one or more stimulants. In some aspects, the one or more stimulants in the beverage can be selected from the group consisting of a methylxanthine, a B vitamin, yerba mate, guarana, ephedra, taurine, carnitine, creatine, sucralose, maltodextrin, acai, glucuronolactone, yohimbine, ginseng, gingko biloba, kola nut, alpinia galanga, L-theanine, mango leaf, ornithine, blackcurrant, ashwagandha, coffeeberry, arginine silicate, curcumin, methylliberine, sideritis scardica, green oat extract, hawthorn, holy basil, lemon balm, Rhodiola rosea, rosemary, sage, spearmint, theacrine, tyrosine, cocoa flavanols, CoQ10, moringa, cacao, guayusa, and any combination thereof. In some aspects, the methylxanthine can be caffeine, theobromine, theophylline, or any combination thereof. In some aspects, the B vitamin can be thiamine, riboflavin, niacin, pantothenic acid, pyridoxine, biotin, folate, cobalamin, cyanocobalamin, inositol, or any combination thereof. In some aspects, the one or more stimulants comprises taurine, glucuronolactone, ginseng, caffeine, a B vitamin, or any combination thereof. In some aspects, the one or more stimulants comprises a combination of taurine, glucuronolactone, ginseng, caffeine, and a B vitamin.

In some aspects, the stimulant is present in the beverage in free form (e.g., powdered form). In some aspects, the stimulant is present in the beverage in an encapsulated form. In some aspects, the beverage can comprise free and encapsulated forms of one or more stimulants.

In some aspects, the beverage (e.g., an energy drink) comprises caffeine as a stimulant. In some aspects, the caffeine is present in the beverage in free form (e.g., powdered form). In some aspects, the caffeine is present in the beverage in an encapsulated form. In some aspects, the beverage can comprise free and encapsulated forms of caffeine.

Any suitable material can be used for encapsulating a stimulant (e.g., caffeine), as long as the encapsulation material is food safe, biodegradable, and able to form a barrier between an internal phase and the stimulant's surroundings. Examples of a suitable encapsulation material include, e.g., a polysaccharide, a protein, a lipid, polyvinylpyrrolidone (PVP), paraffin, shellac, and any combination thereof. In an aspect, the polysaccharide can be a starch, amylose, amylopectin, a dextrin, a maltodextrin, polydextrose, cellulose, gum Arabic, gum tragacanth, gum karaya, mesquite gum, galactomannans, pectin, soluble soybean polysaccharide, carrageenan, alginate (e.g., sodium alginate), dextran, chitosan, xanthan, gellan, or any combination thereof. In an aspect, the protein can be casein, gelatin, gluten, or any combination thereof. In an aspect, the lipid can be any food safe lipid, such as fatty acid, fatty alcohol, wax (e.g., beeswax, carnauba was, candellia wax), a glyceride, a phospholipid, or any combination thereof.

The encapsulated stimulant (e.g., encapsulated caffeine) can be purchased commercially or prepared. For example, the stimulant (e.g., caffeine) can be spray dried, spray-bed dried, fluid-bed coated, spray-chilled, spray-cooled, melt injected, extruded, and/or emulsified along with the encapsulation material to provide an encapsulated stimulant.

Typically, the beverage composition can comprise other components to provide, e.g., sweetness and flavor. In an aspect, the beverage composition can include, for example, a sweetener (e.g., a nutritive or non-nutritive sweetener), a preservative, color, a flavor, an antioxidant, a nutrient, or any combination thereof. In an aspect, the beverage composition can comprise water, one or more stimulants, at least one sweetener, at least one preservative, color, at least one flavor, and at least one vitamin.

In some aspects, the beverage composition can comprise at least one (e.g., 1, 2, 3, 4, etc.) sweetener. In some aspects, the sweetener can be a nutritive sweetener. Typical nutritive sweeteners include, e.g., sugar (i.e., sucrose), dextrose, fructose, high fructose corn syrup, and combinations thereof. In some aspects, the sweetener can be sucrose. In some aspects, the sweetener can be a non-nutritive sweetener, including, for example, a natural non-nutritive sweetener (e.g. a steviol glycoside) and/or an artificial non-nutritive sweetener. Examples of a non-nutritive sweetener include, e.g., acesulfame potassium, advantame, aspartame, neotame, saccharin, sucralose, a steviol glycoside (e.g., stevioside, dulcoside, rebaudioside A, rebaudioside B, rebaudioside C, rebaudioside D, rebaudioside E, rebaudioside F, rebaudioside M), or any combination thereof. In some aspects, the beverage can comprise sucralose, acesulfame potassium, or a combination of both. In some aspects, the beverage can comprise sucralose and acesulfame potassium.

In some aspects, the beverage composition can further comprise at least one (e.g., 1, 2, 3, 4, etc.) preservative. In some aspects, the preservative can be sodium benzoate, potassium benzoate, calcium propionate, potassium sorbate, sodium sorbate, calcium disodium edetate, a sodium polyphosphate (e.g., sodium acid polyphosphate, sodium hexamethaphosphate, sodium tripolyphosphate, tetrasodium pyrophosphate, or sodium trimetaphosphate), or any combination thereof.

In some aspects, the beverage composition can comprise at least one (e.g., 1, 2, 3, 4, etc.) antioxidant. In some embodiments, the antioxidant can be a vitamin (e.g., vitamin A (e.g., retinol, a carotenoid), vitamin C, (e.g., ascorbic acid), vitamin E (e.g., a tocopherol)), a polyphenol (e.g., a flavonoid, a phenolic acid, a lignin, or a stilbene), or any combination thereof. Vitamin E is a group of eight fat soluble compounds that include four tocopherols (i.e., α-tocopherol, β-tocopherol, γ-tocopherol, and δ-tocopherol) and four tocotrienols (i.e., α-tocotrienol, β-tocotrienol, γ-tocotrienol, and δ-tocotrienol).

In some aspects, the beverage composition can comprise at least one (e.g., 1, 2, 3, 4, etc.) nutrient. In some embodiments, the nutrient can be a vitamin, a mineral, an amino acid, a carbohydrate, a lipid, a phytonutrient, protein, fiber, choline, or any combination thereof.

Examples of the mineral include, e.g., calcium, potassium, magnesium, selenium, zinc, phosphorus, iodine, copper, manganese, iron, chlorine, cobalt, molybdenum, and any combination thereof. In some aspects, the mineral can be calcium, potassium, magnesium, phosphorus, or any combination thereof.

Examples of the vitamin include, e.g., vitamin A (e.g., all-trans-retinols, all-trans-retinyl esters, all-trans-beta-carotene, a provitamin A carotenoid), a vitamin B, vitamin C (e.g., ascorbic acid, an ascorbate), vitamin D (e.g., a calciferol), vitamin E (e.g., a tocopherol, a tocotrienol), vitamin K (e.g., a phylloquinone, a menaquinone, or a menadione), or any combination thereof. The B vitamin can be, for example, B₁, B₂, B₃, B₅, B₆, B₇, B₉, B₁₂, or any combination thereof.

Examples of the amino acid include, e.g., alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, any salt thereof, and any combination thereof.

Examples of the carbohydrate include, e.g., a monosaccharide (e.g., hexose, pentose), a polysaccharide (e.g., a beta-glucan, a fructan, a lignin, a pectin), and any combination thereof.

Examples of the lipid include, e.g., a triglyceride (e.g., fat, oil), a fatty acid, a phospholipid (e.g., lecithin), a sterol (e.g., cholesterol, a plant sterol, such as sitosterol), and any combination thereof.

Examples of a phytonutrient include, e.g., a flavonoid (e.g., a flavonol, a flavanone, a flavone, a flavan-3-ol, a flavanonol, an anthocyanidin), a phenolic acid, a hydroxycinnamic acid, a phenylethanoid, a phenolic compound (e.g., a monophenol, a polyphenol), a carotenoid (e.g., a carotene, a xanthophylls, a triterpenoid, a diterpene, a monoterpene, a steroid), an isoflavinoid (e.g., an isoflavone, an isoflavane, an isoflavandiol, an isoflavene, an isoflavene, a pterocarpan, coumestrol), an aurone, a chalconid, a flavonolignan, a lignan, a phytoestrogen, a stilbenoid, a curcuminoid, a tannin, a hydrolysable tannin, a condense tannin, a phlorotannin, a flavono-ellagitannin), a glucosinolate (e.g., an aglycone derivative, an organosulfide, an indole), a betalain (e.g., betacyanin, a betaxanthin), chlorophyllin, capsaicin, gingerol, an alkylresorcinol, piperine, or any combination thereof.

Examples of fiber include, e.g., soluble fiber, such as a plant fiber. Examples of a plant fiber include, e.g., psyllium, a fructan, such as inulin (e.g., cassava root inulin, chicory root inulin), levan, or phlein, or any combination thereof.

Examples of protein include, a water soluble protein, such as a dairy protein, an egg protein, or a plant-based protein. Examples of a dairy protein include, e.g., whey protein. Examples of a plant-based protein include protein extracted from, e.g., soy beans, navy beans, lentil beans, chickpeas, peas, a seed (e.g., cranberry, pumpkin, sunflower, flax, quinoa, chia, hemp, rapeseed, canola), whey, alfalfa, almonds, peanuts, rice, and any combination thereof.

In some aspects, the beverage composition can comprise at least one (e.g., 1, 2, 3, 4, etc.) flavoring agent. The flavoring agent can be any compatible food safe agent used for flavoring foods or beverages. The flavoring agent can be natural or synthetic. Non-limiting examples include, e.g., for example, a citrus flavor (e.g., limonene, octanal), a vanilla flavor (e.g., vanilla extract, vanillin), a cinnamon flavor (e.g., cinnamic acid), a fruit flavor (e.g., cherry, raspberry, strawberry, grape, strawberry, pineapple, passionfruit), and any combination thereof.

In some aspects, the beverage further comprises water and at least one additive selected from a sweetener, a preservative, color, a flavor, an antioxidant, a nutrient, and any combination thereof. In some aspects, the beverage further comprises water and at least one nutrient.

In the method, the beverage is administered to a consumer. In an aspect, the administering step comprises the consumer orally ingesting the beverage. In some aspects, consumption of the beverage (e.g., a 500 ml beverage) will be completed within 15 min from the start (time zero). In some aspects, approximately one-third of the beverage (e.g., a 500 ml beverage) can be consumed about every 5 min.

In some aspects, the consumer will have refrained from caffeine consumption for 48 hours prior to the start of the method. In some aspects, the consumer will have refrained from caffeine consumption for 48 hours and fasted for 10 hours prior to the start of the method. In some aspects, the consumer will have (i) refrained from caffeine consumption for 48 hours, (ii) fasted for 10 hours, (iii) refrained from alcohol for 24 hours, and (iv) refrained from nicotine for 48 hours prior to the start of the method.

After the beverage has been administered, the consumer's plasma concentration and VAS response are measured. These steps can be measured in either order and can be measured simultaneously (or near simultaneously) or intermittently. In an aspect, the VAS response (when measured) can be measured within about 10 min (e.g., within about 5 min) after a blood draw to measure the plasma concentration.

In an aspect, a blood sample (e.g., a venous blood sample) of the consumer can be evaluated over a first time period to determine the plasma concentration of the one or more stimulants. In some aspects, the first time period is about 30 minutes to about 15 hours (e.g., about 30 min to 14 h, about 30 min to 13 h, about 30 min to 12 h, about 30 min to 11 h, about 30 min to 10 h, about 30 min to 9 h, about 30 min to 8 h, about 30 min to 7 h, about 30 min to 6 h, about 30 min to 5 h, about 30 min to 4 h, about 30 min to 3 h, about 30 min to 2 h, or about 30 min to 1 h) after the beverage has been administered (e.g., orally ingested). The first sip of beverage administration is counted as time zero. In some aspects, the first time period is 30 minutes to 12 hours after the beverage has been administered.

In some aspects, the consumer's plasma concentration is measured about every 30 minutes during the first time period. In an example, blood sampling timepoints in the first time period can be, e.g., 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, and 12 hours after the first sip of beverage ingestion. In an aspect, the blood sample window can be 30 min to 6 hours±2 min and 7 to 12 hours±5 min.

The amount of “energy” provided by the beverage comprising one or more stimulants can be measured by subjectively measuring factors, such as alertness and tiredness. While not wishing to be bound by theory, it is believed that alertness relates more to energy of the mind, whereas tiredness related more to energy of the body. It has been found that alertness and tiredness are inversely related, e.g., as alertness increases, tiredness decreases.

Accordingly, before or after the plasma concentration has been measured, the consumer's visual analog scale (VAS) response can be measured in a second time period. The VAS response is a subjective measure of a consumer's physical and mental experience after (and optionally before) consuming the beverage. In general, the VAS consists of a line (e.g., about a 10 cm line) with verbal anchors at either end. To provide a response, the subject places a mark at a point on the line corresponding to the subject's rating of each factor. In an example, a consumer is asked to evaluate the level at the moment the test is administered for the following factors: alert (ranging from not at all to extremely), headache (ranging from no headache to extreme headache), irritable (ranging from not at all to extremely), jittery (ranging from not at all to extremely), lightheaded (ranging from not at all to extremely), overall mood (ranging from very bad to very good), relaxed (ranging from not at all to extremely), tense (ranging from not at all to extremely), tired (ranging from not at all to extremely) and pounding heart (ranging from not at all to extremely). In some aspects, the consumer's VAS response comprises measuring one or more factors selected from alertness, headache, irritability, jitteriness, lightheadedness, overall mood, relaxation, tension, tiredness, or any combination thereof. In some aspects, the consumer's VAS response comprises measuring alertness. In some aspects, the consumer's VAS response comprises measuring alertness and tiredness.

VAS responses can be numerically quantified, and in some cases, one or more responses can be combined to provide an overall VAS response. In some aspects, to quantify mood ratings of certain factors (e.g., alertness) from the VAS response after consumption of an EC beverage can be compared to those for an IRC beverage in a consumer (or compared between consumers in both groups).

In some aspects, the second time period is about 30 minutes to about 15 hours (e.g., about 30 min to 14 h, about 30 min to 13 h, about 30 min to 12 h, about 30 min to 11 h, about 30 min to 10 h, about 30 min to 9 h, about 30 min to 8 h, about 30 min to 7 h, about 30 min to 6 h, about 30 min to 5 h, about 30 min to 4 h, about 30 min to 3 h, about 30 min to 2 h, or about 30 min to 1 h) after the beverage has been administered (e.g., orally ingested). The first sip of beverage administration is counted as time zero. In some aspects, the second time period is 30 minutes to 12 hours after the beverage has been administered.

In some aspects, the consumer's VAS response is measured about every 60 minutes during the second time period. In an example, the VAS response timepoints in the second time period can be, e.g., 1, 2, 3, 4, 5, 6, 8, 10, and 12 hours after the first sip of beverage ingestion.

In some aspects, the first and second time periods are different lengths of time. In some aspects, the first and second time periods are the same length of time but include measurements on different time scales (e.g., intermittent measurements).

In some aspects, the method further comprises measuring a baseline for each of the consumer's plasma concentration and VAS response prior to administering the beverage (step (a)). In some aspects, the baseline is measured at about 5 to 60 minutes (e.g., about 10 to 60 min, about 15 to 60 min, about 20 to 60 min, about 5 to 30 min, about 10 to 30 min, about 15 to 30 min, about 20 to 30 min, about 5 min, about 10 min, about 15 min, about 20 min, about 25 min, or about 30 min) prior to consuming the beverage. In some aspects, the baseline is measured at about 5 to 30 minutes prior to (a). In some aspects, the baseline for each parameter is measured within about 30 minutes prior to consuming the beverage.

In some aspects, the method can further comprise measuring one or more vital signs of the consumer prior to (d) in a third time period. In some aspects, the one or more vital signs comprises heart rate, systolic blood pressure, diastolic blood pressure, or any combination thereof. In some aspects, the consumer's heart rate, systolic blood pressure, and diastolic blood pressure are measured.

In some aspects, the third time period can be about 30 minutes to about 15 hours (e.g., about 30 min to 14 h, about 30 min to 13 h, about 30 min to 12 h, about 30 min to 11 h, about 30 min to 10 h, about 30 min to 9 h, about 30 min to 8 h, about 30 min to 7 h, about 30 min to 6 h, about 30 min to 5 h, about 30 min to 4 h, about 30 min to 3 h, about 30 min to 2 h, or about 30 min to 1 h) after the beverage has been administered (e.g., orally ingested). The first sip of beverage administration is counted as time zero. In some aspects, the third time period is 30 minutes to 12 hours after the beverage has been administered.

In some aspects, the consumer's one or more vital signs can be measured about every 2 to 4 hours during the third time period. In some aspects, when the vital sign measurement coincides with a blood draw for a plasma concentration measurement, the vital sign measurement can be performed within about 15 min after the blood draw. In an example, the vital sign timepoints in the third time period can be, e.g., 2 and 6 hours after the first sip of beverage ingestion.

In some aspects, the first, second, and third time periods are different lengths of time. In some aspects, the first, second, and third time periods are the same length of time but include measurements on different time scales (e.g., intermittent measurements).

In an aspect, the consumer's one or more vital signs can be measured prior to administering the beverage (step (a)). In some aspects, the baseline is measured at about 5 to 30 minutes (e.g., about 10 to 30 min, about 15 to 30 min, about 20 to 30 min, about 5 min, about 10 min, about 15 min, about 20 min, about 25 min, or about 30 min) prior to consuming the beverage. In some aspects, the baseline for the vital signs can be measured within about 30 minutes prior to consuming the beverage.

In some aspects, the consumer can be administered one or more meals and/or snacks during a fourth time period. The meal and/or snack can be any suitable food and should be consumed within about 30 min (e.g., within about 20 min) after being eating has begun. In some aspects, two meals and one optional snack are administered during the fourth time period. In some aspects, two meals and one snack are administered during the fourth time period.

In some aspects, the fourth time period can be about 2 hours to about 15 hours (e.g., about 2 to 14 h, about 2 to 13 h, about 2 to 12 h, about 2 to 11 h, about 2 to 10 h, about 2 to 9 h, about 2 to 8 h, about 2 to 7 h, about 2 to 6 h, about 2 to 5 h, about 2 to 4 h, or about 2 to 3 h) after the beverage has been administered (e.g., orally ingested). The first sip of beverage administration is counted as time zero. In some aspects, the fourth time period is about 4 to 9 hours after the beverage has been administered.

In some aspects, a meal and/or snack can be administered about every 3 to 5 hours during the fourth time period. In some aspects, when the meal and/or snack coincides with a blood draw for a plasma concentration measurement, the meal and/or snack can be administered within about 30 min (e.g., within about 20 min or within about 15 min) of the blood draw. In an example, the meal and/or snack timepoints in the fourth time period can be, e.g., 4 (meal), 9 (meal), and optionally 12 (snack) hours after the first sip of beverage ingestion.

Once the plasma concentration and VAS response has been measured, the amount of the one or more stimulants in the beverage can be adjusted, as needed, to provide sustained energy when the beverage is ingested by a consumer. For example, the calculated concentration of caffeine at any time in the plasma (i.e., the plasma caffeine concentration) can be directly correlated to the manifestation of the caffeine as measured via VAS response. In some aspects, the amount of the one or more stimulants in the beverage can be increased in the beverage based on the measurements from the plasma concentration (step (b)) in combination with the VAS response (step (c)). In some aspects, the amount of the one or more stimulants in the beverage can be decreased in the beverage based on the measurements from the plasma concentration (step (b)) in combination with the VAS response (step (c)).

In some aspects, the amount of the one or more stimulants in the beverage is increased based on a desired VAS response. In some aspects, the amount of the one or more stimulants in the beverage is decreased based on a desired VAS response.

In some aspects, the amount of the one or more stimulants in the beverage is increased based on a desired vital sign measurement. In some aspects, the amount of the one or more stimulants in the beverage is decreased based on a desired vital sign measurement.

In some aspects, the amount of the one or more stimulants in the beverage is increased based on consumer preference data. In some aspects, the amount of the one or more stimulants in the beverage is decreased based on consumer preference data.

The adjustment in the amount of the at least one stimulant can be incremental, such as ±1 mg, ±5 mg, ±10 mg, ±15 mg, ±25 mg, +50 mg, +75 mg, or +100 mg. In general, the adjustment (e.g., in a 500 ml beverage) will be such that the total amount of stimulants will be less than 400 mg. For example, the total amount of stimulants can be about 400 mg or less, about 390 mg or less, about 380 mg or less, about 370 mg or less, about 360 mg or less, about 350 mg or less, about 340 mg or less, about 330 mg or less, about 320 mg or less, about 310 mg or less, about 300 mg or less, about 290 mg or less, about 280 mg or less, about 270 mg or less, about 260 mg or less, about 250 mg or less, about 240 mg or less, about 230 mg or less, about 220 mg or less, about 210 mg or less, about 200 mg or less, about 190 mg or less, about 180 mg or less, about 170 mg or less, about 160 mg or less, about 150 mg or less, about 140 mg or less, about 130 mg or less, about 120 mg or less, about 110 mg or less, about 100 mg or less, about 90 mg or less, about 80 mg or less, or about 70 mg or less.

In some aspects, the total concentration of stimulants in a beverage (e.g., a 500 ml beverage) will be about 50 mg to about 400 mg (e.g., about 50 mg to about 380 mg, about 50 mg to about 360 mg, about 50 mg to about 340 mg, about 50 mg to about 320 mg, about 50 mg to about 300 mg, about 50 mg to about 280 mg, about 50 mg to about 260 mg, about 50 mg to about 250 mg, about 50 mg to about 240 mg, about 50 mg to about 220 mg, about 50 mg to about 200 mg, about 50 mg to about 180 mg, about 50 mg to about 160 mg, about 50 mg to about 140 mg, about 50 mg to about 120 mg, about 50 mg to about 100 mg, about 50 mg to about 80 mg, about 50 mg to about 60 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 250 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 250 mg, about 100 mg to about 240 mg, about 00 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 250 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 150 mg to about 400 mg, about 150 mg to about 380 mg, about 150 mg to about 360 mg, about 150 mg to about 340 mg, about 150 mg to about 320 mg, about 150 mg to about 300 mg, about 150 mg to about 280 mg, about 150 mg to about 260 mg, about 150 mg to about 250 mg, about 150 mg to about 240 mg, about 150 mg to about 220 mg, about 150 mg to about 200 mg, about 150 mg to about 180 mg, about 150 mg to about 160 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 250 mg, about 180 mg to about 240 mg, about 180 mg to about 220 mg, about 180 mg to about 200 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 250 mg, about 200 mg to about 240 mg, about 200 mg to about 220 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 250 mg, about 220 mg to about 240 mg, about 250 mg to about 400 mg, about 250 mg to about 380 mg, about 250 mg to about 360 mg, about 250 mg to about 340 mg, about 250 mg to about 320 mg, about 250 mg to about 300 mg, about 250 mg to about 280 mg, about 250 mg to about 260 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 400 mg, about 300 mg to about 380 mg, about 300 mg to about 360 mg, about 300 mg to about 340 mg, about 300 mg to about 320 mg, about 350 mg to about 400 mg, about 350 mg to about 380 mg, about 350 mg to about 360 mg, or about 380 mg to about 400 mg.

In some aspects, the amount of the one or more stimulants in the beverage can be increased or decreased in the beverage to provide a beverage with sustained energy (i.e., consistent or long lasting energy that does not vary more than +15% or more than ±10%) for about 2 hours or more (e.g., about 3 hours or more, about 4 hours or more, about 5 hours or more, about 6 hours or more, about 7 hours or more, about 8 hours or more, about 9 hours or more, about 10 hours or more, about 11 hours or more, or about 12 hours or more) after the beverage has been administered. For example, the energy level can be measured via VAS at the baseline and at any point throughout the measurement period to determine if energy has increased and the increase has been maintained for a certain period of time. In some aspects, the amount of the one or more stimulants in the beverage can be increased or decreased in the beverage to provide a beverage with sustained energy (i.e., consistent energy; long lasting energy) for up to 12 hours (e.g., up to 11 hours, up to 10 hours, up to 9 hours, up to 8 hours, up to 7 hours, up to 6 hours, up to 5 hours, up to 4 hours, or up to 3 hours) after the beverage has been administered. The sustained energy can be measured in accordance with the method described herein, including measuring alertness and tiredness in a consumer post-ingestion and compared to a baseline for each factor prior to consumption (e.g., within 30 min prior to consumption).

In an aspect, the method provides sustained energy from a beverage comprising one or more stimulants, as described herein, with reduced adverse effects (e.g., reduced incidence of headache, jitteriness, and/or pounding heart) compared to a beverage comprising a different concentration of one or more stimulants, a different combination of one or more stimulants, or both.

In an aspect, the method of providing sustained energy from a beverage comprising one or more stimulants, comprises:
(a) measuring a baseline for a consumer's (i) plasma concentration and (ii) visual analog scale (VAS) response;
(b) administering the beverage comprising one or more stimulants to the consumer; (c) measuring the consumer's plasma concentration of the one or more stimulants over a first time period;
(d) measuring the consumer's VAS response over a second time period; and
(e) increasing or decreasing the amount of the one or more stimulants in the beverage based on the measurements from (c) and (d) to provide sustained energy for about 2 hours or more (e.g., about 5 hours or more).

In an aspect, the method of providing sustained energy from a beverage comprising one or more stimulants, comprises:
(a) measuring a baseline for a consumer's (i) plasma concentration and (ii) visual analog scale (VAS) response;
(b) administering the beverage comprising one or more stimulants to the consumer; (c) measuring the consumer's plasma concentration of the one or more stimulants over a first time period;
(d) measuring the consumer's VAS response over a second time period;
(e) measuring one or more vital signs of the consumer prior in a third time period; and
(f) increasing or decreasing the amount of the one or more stimulants in the beverage based on the measurements from (c), (d), and optionally (e) to provide sustained energy for about 2 hours or more (e.g., about 5 hours or more).

Aspects of the beverage composition, measuring steps, including the first, second, and third time periods, and adjusting the amount of stimulant(s) in the beverage are as described herein.

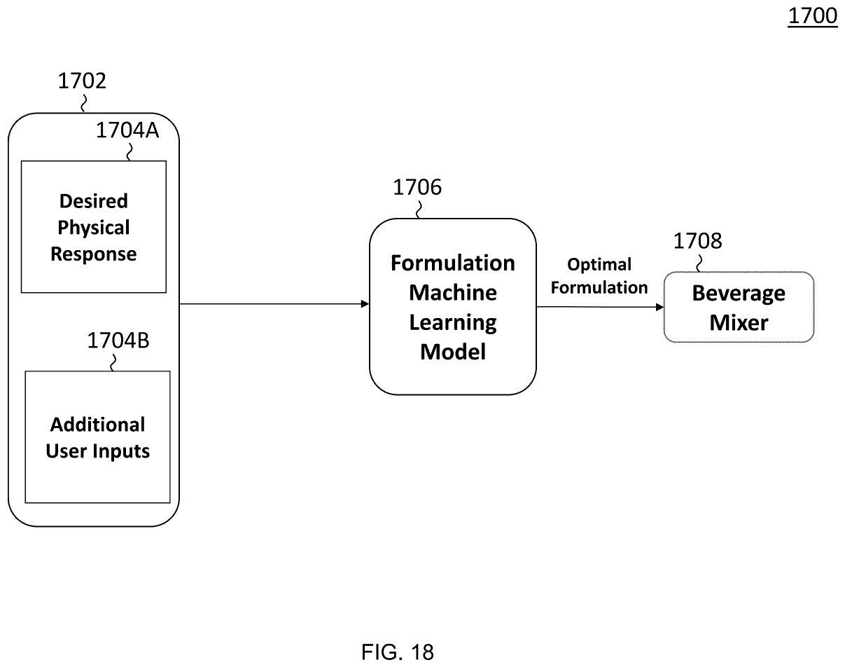

FIG. 18 is a diagram of an example formulation optimization system 1700 for generating optimal formulations of stimulants for a beverage, including an EC and IRC beverage using a trained machine learning model. In some aspects, the formulations may include any combination of one or more stimulants. In some aspects, the amount of the one or more stimulants may be determined using a trained machine learning model based on a desired physical response, such as a desired VAS response or a desired vital sign measurement. For example, the trained machine learning model may be trained to output different formulations of the stimulants within the beverage optimized to achieve different desired VAS responses. Formulations may include amounts of the stimulant and identification of different types of stimulant (e.g., the one or more stimulants in the beverage can be selected from the group consisting of a methylxanthine, a B vitamin, yerba mate, guarana, ephedra, taurine, carnitine, creatine, sucralose, maltodextrin, acai, glucuronolactone, yohimbine, ginseng, gingko biloba, kola nut, alpinia galanga, L-theanine, mango leaf, ornithine, blackcurrant, ashwagandha, coffeeberry, arginine silicate, curcumin, methylliberine, sideritis scardica, green oat extract, hawthorn, holy basil, lemon balm, Rhodiola rosea, rosemary, sage, spearmint, theacrine, tyrosine, cocoa flavanols, CoQ10, moringa, cacao, guayusa, and any combination thereof). In some aspects, the one or more stimulants may further comprise taurine, glucuronolactone, ginseng, caffeine, a B vitamin, or any combination thereof.

In general, a desired VAS response may be specified, as described above, as a line graph with verbal anchors at either end (e.g., “none,” “not at all,” and “very bad” on the far left and “extreme, “extremely,” and “very good” on the far right). The desired VAS response may further comprise one or more factors for achieving a desired consumer experience, such as alertness, headache, irritability, jitteriness, lightheadedness, overall mood, relaxation, tension, tiredness, or any combination thereof.

In some aspects, formulation optimization system 1700 may include an input device 1702, a formulation machine learning model 1706, and a beverage mixer 1708. Only one input device 1702, one formulation machine learning model 1706, and one beverage mixer 1708 is depicted for simplicity. It is understood that formulation optimization system 1700 may include any number of input devices, formulation machine learning models, and beverage mixers. In some aspects, formulation optimization system 1700 may be implemented in a distributed manner such as in different locations. For example, input device 1702 may be implemented in one location, formulation machine learning model 1706 in another location, and beverage mixer 1708 in yet another location, and communication within formulation optimization system 1700 would be transmitted over a network. In some aspects, one or more components of formulation optimization system 1700 may be co-located with each other, such as in a manufacturing facility.

Input device 1702 is configured to provide inputs to formulation machine learning model 1706, which is configured to generate one or more optimized formulations of a stimulant for a beverage based on the provided inputs. In some aspects, the terms “optimized formulation” or “optimal formulation” refers to a particular combination of ingredients (e.g., stimulants) that is most therapeutically effective in achieving the desired physical response (e.g., VAS response, vital sign response). As noted above, the formulation may specify specific amounts of stimulants and specific types of stimulants and different formulations may be generated based on the desired physical response. That is, there may be different optimized formulations for different physical responses. In some aspects, the term “optimized formulation” or “optimal formulation” refers to a particular combination of ingredients (e.g., stimulants) that is one or more of: most therapeutically effective in providing a desired physical response (e.g., a certain threshold of increased alertness and decreased tiredness over a certain period of time) and most therapeutically effective in achieving a consumer preference. In some aspects the desired physical response may be provided as threshold values for the particular physical response, such as heart rate, alertness, etc., and for a specific period of time, such as certain number of hours.

In some aspects, input device 1702 may provide a desired physical response 1704A as input to formulation machine learning model 1706. Examples of desired physical response 1704A included desired VAS responses and desired vital sign measurements. In some aspects, input device 1702 may provide additional user inputs 1704B in addition to the desired VAS response 1704A. In some aspects, only desired VAS response 1704A is provided as input. In some aspects, only additional user inputs 1704B may be provided as input. Additional user inputs 1704B may include consumer preference data and/or other desired characteristics of beverage such as, but not limited to, flavor, consistency, color, aroma, texture, and aftertaste, just to name a few examples. In some aspects, additional user inputs 1704B may also be factored by formulation machine learning model 1706 when generating an optimized formulation of ingredients for the beverage.

In some aspects, additional user inputs 1704B may be measured data from one or more users such as a measured plasma concentration of the amount of one or more stimulants over a first time period and a measured VAS response over a second time period. These measured amounts may be collected after administering the beverage comprising an amount of one or more stimulants to a consumer. Formulation machine learning model 1706 may be trained to generate a stimulant amount based on the measured plasma concentration and the measured VAS response. This generated stimulant amount may then be used to increase or decrease the amount of the one or more stimulants in the beverage to match the stimulant amount. In this manner, formulation machine learning model 1706 may be utilized to determine a stimulant amount based on measured physical responses.

In some aspects, formulation machine learning model 1706 is trained based on test data (e.g., collected from one or more clinical studies) and is trained to output optimal formulations based on this test data. In some aspects, formulation optimization system 1700 may include one formulation machine learning model 1706. The test data may be processed by formulation machine learning model 1706 as part of generating the optimal formulations. One example of processing includes feature engineering and selection which transforms the collected raw data into a format that is more suitable for generating the optimized formulations. Formulation machine learning model 1706 may extract key attributes from the raw data on ingredient combinations and physical responses. For instance, features such as ingredient proportions and physical responses may be quantified. Feature selection algorithms, like Recursive Feature Elimination (RFE) or Lasso regression, may then be applied to identify and retain the most predictive features within the raw data.

In some aspects, training of the formulation machine learning model 1706 may utilize a supervised learning approach, where formulation machine learning model 1706 is trained using raw data comprising specific combinations and quantities of ingredients paired with documented physical responses from test subjects. Trained in this manner, formulation machine learning model 1706 may employ regression analysis to generate stimulant combinations based on a desired VAS response.

In some aspects, training of the formulation machine learning model 1706 may utilize unsupervised learning techniques. In these aspects, formulation machine learning model 1706 may explore the raw dataset of various ingredient combinations and their associated physical responses without pre-labeled outcomes. Clustering algorithms, such as k-means or hierarchical clustering, may be employed to identify patterns and natural groupings in the data, revealing insights into which combinations of stimulants are most effective (i.e., optimal) for certain physical VAS responses.

In some aspects, formulation optimization system 1700 may include two or more formulation machine learning models, each being trained for different purposes, such as for different types of beverages (e.g., EC, TRC) or different groups of users (e.g., teens, adults). For example, there may be a formulation machine learning model 1706 trained specifically for a group of users and therefore is configured to generate optimized formulations for that particular group of users. As another example, there may be yet another formulation machine learning model 1706 trained specifically for stimulant beverages and therefore is configured to generate optimized formulations for stimulant beverages.

Formulation machine learning model 1706 is trained to provide optimized ingredient combination and quantification with the ability to determine not just the types of stimulants but also their optimal amounts for desired VAS responses.

In some aspects, formulation machine learning model 1706 may be implemented as any type of machine learning model such as, but not limited to, a neural network, random forests, gradient boosting machines, and clustering algorithms. For example, if implemented with a neural network architecture, formulation machine learning model 1706 may be configured to perform regression analysis of stimulant combinations and amounts, and their VAS responses. Other types of machine learning models are possible and may be used instead of or in combination with (i.e., a hybrid implementation). For example, a random forest implementation provides improved capability for handling complex, non-linear relationships between the stimulants and VAS responses that may be present in the provided raw data.

In some aspects, formulation machine learning model 1706 may perform time-series analysis for generating an optimized formulation based on a desired VAS response. Time-series analysis can be used to predict the temporal relationship between VAS response and stimulants, post-consumption. This allows for the optimization of stimulant formulation based on not only their efficacy but also their onset and duration of effect.

In some aspects, formulation machine learning model 1706 may be implemented with clustering algorithms (e.g., k-means, hierarchical clustering) which are useful for grouping similar data points (e.g., stimulant combinations and their VAS responses) and can help in identifying patterns within the raw data. Clustering algorithms can be beneficial for organizing combination of stimulants into categories based on their effectiveness and the type of physical VAS response they induce.

In some aspects, formulation machine learning model 1706 may be implemented with autoencoders, which a type of neural network used for unsupervised learning. These neural network models are useful for learning efficient codings of the input data. Autoencoders can learn to identify which stimulants (or combinations thereof) are most relevant for specific VAS responses by detecting complex, non-linear relationships between different stimulants. This can be particularly useful when the effect of a stimulant is not linear or when the interactions between multiple stimulants are complex. Autoencoders can also be effective in filter out irrelevant or misleading information which can help in focusing on stimulants that have more direct and significant impact on the desired VAS responses. In some aspects, formulation machine learning model 1706 may implement variational autoencoders, which could be useful in generating new stimulation combinations that were not present in the original raw data. That is, after training on existing combinations of stimulants from the raw data, formulation machine learning model 1706 with a variational autoencoder can be used to generate new, potentially effective ingredient combinations that have not been explicitly observed in the raw data.

Returning to FIG. 18, formulation machine learning model 1706 may provide the optimized formulation to beverage mixer 1708, which is configured to blend various ingredients, including stimulants based on the optimized formulation, to produce the beverage. In this manner, the beverage from beverage mixer 1708 is produced based on the optimized formulation. Beverage mixer 1708 may be configured to combine the necessary ingredients along with the specified amounts and types of stimulants in the optimized formulation.

In an aspect, the method of providing sustained energy from a beverage comprising one or more stimulants comprises (a) administering the beverage comprising an amount of one or more stimulants to a consumer; (b) measuring the consumer's plasma concentration of the amount of one or more stimulants over a first time period; (c) measuring the consumer's visual analog scale (VAS) response over a second time period; (d) generating, using a trained machine learning model, a stimulant amount based on the measurements from (b) and (c); and (e) increasing or decreasing the amount of the one or more stimulants in the beverage to match the stimulant amount.
(a) In some aspects, the machine learning model receives, as input, a desired VAS response and is configured to output an optimal formulation of stimulants for achieving the desired VAS response in the beverage. The formulation of stimulants may specified one or more types of stimulants and the amounts of the one or more types of stimulants to be included when producing the beverage.

In some aspects, the optimal formulation may be provided to a beverage mixer for producing a beverage with the specified amounts and types of stimulants.

In some aspects, there may be more than one machine learning model implemented in a formulation optimization system. For example, there may be different machine learning models trained to output different optimal formulations for different types of beverages and/or different types of users (e.g., teens, adults).

In some aspects, the machine learning model is used to determine the optimal amount of stimulant based on a desired VAS response. In some aspects, the machine learning model is used to determine the optimal combination of stimulants based on a desired VAS response.

In some aspects, the machine learning model is used to determine an optimal amount of stimulant based on a desired vital sign measurement. In some aspects, the machine learning model is used to determine the optimal combination of stimulants based on a desired vital sign measurement.

In some aspects, the machine learning model is used to determine an optimal amount of stimulant based on consumer preference data. In some aspects, the machine learning model is used to determine the optimal combination of stimulants based on consumer preference data.

In some aspects, the machine learning model is used to determine the optimal value for the first time period or the second time period.

In some aspects, the present disclosure relates to a method of providing sustained energy from a beverage comprising one or more stimulants, the method comprising: (a) providing user input to a formulation machine learning model, wherein the formulation machine learning is trained to generate an optimized formulation based on the user input, and wherein the user input comprises a desired VAS response; (b) outputting, by the formulation machine learning model, the optimized formulation, wherein the optimized formulation comprises a combination of the one or more stimulants including at least one of an amount of the one or more stimulants and a type of the one or more stimulants; and (c) providing the optimized formulation to a beverage mixer for producing the beverage based on the combination of the one or more stimulants.

In some aspects of this method, the one or more stimulants is selected from the group consisting of a methylxanthine, a B vitamin, yerba mate, guarana, ephedra, taurine, carnitine, creatine, sucralose, maltodextrin, acai, glucuronolactone, yohimbine, ginseng, gingko biloba, kola nut, alpinia galanga, L-theanine, mango leaf, ornithine, blackcurrant, ashwagandha, coffeeberry, arginine silicate, curcumin, methylliberine, sideritis scardica, green oat extract, hawthorn, holy basil, lemon balm, Rhodiola rosea, rosemary, sage, spearmint, theacrine, tyrosine, cocoa flavanols, CoQ10, moringa, cacao, guayusa, and any combination thereof. In some aspects, the methylxanthine is caffeine, theobromine, theophylline, or any combination thereof. In some aspects, the B vitamin is thiamine, riboflavin, niacin, pantothenic acid, pyridoxine, biotin, folate, cobalamin, cyanocobalamin, inositol, or any combination thereof. In some aspects, the one or more stimulants comprises caffeine.

In some aspects, the beverage further comprises water and at least one additive selected from a sweetener, a preservative, color, a flavor, an antioxidant, a nutrient, and any combination thereof.

In some aspects, the combination of the one or more stimulants comprises one stimulant, wherein the optimized formulation comprises at least one of an optimized amount of the one stimulant and a type of the one stimulant, and where the desired physical response is a desired VAS response.

In some aspects, the combination of one or more stimulants comprises a plurality of stimulants, wherein the optimization formulation comprises at least one of optimized amounts of the plurality of stimulants and types of the plurality of stimulants, and wherein the desired physical response is a desired VAS response. In some aspects, the combination of one or more stimulants comprises one stimulant, wherein the optimized formulation comprises at least one of an optimized amount of the one stimulant and a type of the one stimulant, and wherein the desired physical response is a desired vital sign measurement. In some aspects, the combination of one or more stimulants comprises a plurality of stimulants, wherein the optimization formulation comprises at least one of optimized amounts of the plurality of stimulants and types of the plurality of stimulants, and wherein the desired physical response is a desired vital sign measurement. In some aspects, the combination of one or more stimulants comprises one stimulant, wherein the optimized formulation comprises at least one of an optimized amount of the one stimulant and a type of the one stimulant, and wherein the user input further comprises consumer preference data. In some aspects, combination of one or more stimulants comprises a plurality of stimulants, wherein the optimization formulation comprises at least one of optimized amounts of the plurality of stimulants and types of the plurality of stimulants, and wherein the machine learning model is used to determine the optimal combination of stimulants based on consumer preference data.

### EXAMPLES

The example presented below is provided for the purpose of illustration only and the embodiments described herein should in no way be construed as being limited to this example. Rather, the embodiments should be construed to encompass any and all variations which become evident as a result of the teaching provided herein.

### Example 1

In this example, the pharmacokinetic profile of a caffeine containing beverage and associated mood and physiological effects were evaluated. To establish “Mood,” the “alertness” ratings from the Caffeine Research visual analog scale (VAS) were quantified after oral consumption of encapsulated caffeine (EC) and immediate-release (free form) caffeine (IRC) beverages. Pharmacokinetic (PK) parameters including, area under the concentration curve for plasma caffeine (AUC₀₋ₜ, AUC_{0-inf}), peak caffeine concentration (Cₘₐₓ), time to maximal plasma caffeine concentration (Tₘₐₓ), half-life (t_{1/2}), and plasma caffeine concentration by time profile were measured after oral consumption of EC or IRC beverages. Four sample types of caffeinated, carbonated energy beverages were prepared.

Each carbonated beverage was 0 kcal per 500 ml (16.9 oz) serving and contained water (93.6%), energy components (taurine+glucuronolactone+ginseng+vitamin premix+caffeine) (0.77%), sweeteners (sucralose and acesulfame potassium) (0.33%), preservatives (0.2%), colors (0.4%), and flavors (0.74%). The amount of caffeine in each sample type was varied as follows.
160 mg immediate-release caffeine (IRC—control)
160 mg encapsulated caffeine (EC—inventive)
250 mg immediate-release caffeine (IRC—control)
250 mg encapsulated caffeine (EC—inventive)

The IRC beverages contained caffeine in powdered form, whereas the EC beverages contained a combination of free caffeine and encapsulated caffeine. The EC beverages had immediate-release (IR) and encapsulated caffeine (100 mg IRC/150 mg EC in the 250 mg sample; 80 mg IRC/80 mg EC in the 160 mg sample).

The test subjects were healthy men and women that were 18-55 years of age with a BMI between 18 and 32.49 (inclusive) kg/m², who typically consume 1-3 caffeinated beverages/day (not to exceed 400 mg/day). N=77 subjects.

Test subject exclusion criteria included the following.

Reported history or clinical manifestations of significant metabolic (including type 1 or type 2 diabetes mellitus), hepatic, renal, hematological, pulmonary, cardiovascular, gastrointestinal, urological, neurological, or psychiatric disorders unless determined to be clinical not significant by investigator.

History of alcoholism or drug addiction within 1 year prior to Screening, or current alcohol or drug use that, in the opinion of the investigator, will interfere with the subject's ability to comply with the dosing schedule and study evaluations.

Extreme dietary habits, including but not limited to intentional consumption of a high fiber diet, gluten-free, low-carb, vegan, ketogenic.

Pregnancy or breastfeeding or planning to become pregnant.

Use of any medication known to have an interaction with caffeine including contraceptives (e.g., medications metabolized via the CYP1A2 pathway).

More than 1 tobacco-containing or nicotine-containing product occasions per month on average, or use of such products within 48 hours prior to dosing of each study period.

Participation in any clinical trial within the past 30 days.

Subjects who, in the opinion of the investigator, are unable or unlikely to comply with the dosing schedule and study evaluations.

As part of the screening visit: (1) general health, resting blood pressure, resting heart rate, height, weight; (ii) questionnaire to understand typical caffeine consumption (timing and sources), and (iii) familiarization with Caffeine Research VAS and Other Symptom VAS.

The evaluation included 4 test day visits with minimum washout 7 days. Subjects were instructed to refrain from dietary foods, beverages, medications (including over the counter medications), and supplement containing caffeine for 48 hours prior to each test day visit, nicotine 48 hours prior to each visit, and alcohol for 24 hours prior to each visit. Verbal confirmation of these instructions was required.

Subjects arrived in the morning, fasted for at least 7-9 hours (10-12 hours prior to dosing). Water was permitted up until 1 hour prior to dosing. All subjects were tested to confirm a negative SARS-CoV-2 rapid detection antigen test (regardless of vaccine status) at the time of each period check-in. Pre-dose blood caffeine level was assessed. Baseline vitals, such as heart rate (HR) and blood pressure (BP), were measured. The Baseline Caffeine Research VAS and Other Symptoms VAS were administered. Time 0 fasting plasma caffeine blood sample (−30 to immediately prior to first sip). Consumption of the assigned product started at 0 min, and subjects were asked to consume approximately ⅓ of the total beverage every 5 minutes until complete (15 minutes maximum).

Blood sampling timepoints, for plasma caffeine, were: 0.5, 1, 1.5, 2, 2.5, 3, 3.5, 4, 4.5, 5, 5.5, 6, 7, 8, 9, 10, 11 and 12 hours after the first sip of beverage ingestion. Blood sample window: 30 min to 6 hours±2 min; 7 to 12 hours±5 min

Vitals (HR, BP) measured at: baseline after blood sampling, and 2 and 6 hours after the first sip of beverage ingestion and after blood sampling is complete for that time-point.

Caffeine Research VAS and Other Symptoms VAS were assessed at baseline and 1, 2, 3, 4, 5, 6, 8, 10, and 12 hours post beverage ingestion. The surveys were administered immediately following the collection of blood sampling and/or vital sign measurements at the respective timepoints. Immediately after the hour 4 blood sample and completion of both VAS questionnaires, a standardized meal was provided. Subjects were instructed that the meal should be consumed within 20 minutes. At post-dose time points when multiple activities were occurring, the order of collection was: blood sample collection, VAS collection, and then Vital Signs collection. Visual Analog Scales (VAS) and Vital Signs were collected as close to the scheduled time as possible. VAS ratings began within 5 minutes of blood draw. Vital signs were checked within 15 minutes of blood draw.

Subjects were instructed to be in a seated or semi-recumbent position for at least 4 hours after dosing. After 4 hours, subjects will be allowed to engage in normal activities but avoid severe physical and mental exertion. Subjects were allowed to lie down during an adverse event per the discretion of the Investigator and/or the attending physician.

A second standardized meal was provided at hour 9 post-dose. Following the final VAS assessments at hour 12, subjects consumed a snack.

A repeated measures Analysis of Covariance (ANCOVA) in Proc MIXED of SAS version 9.1.3 was used to evaluate the post-dose VAS alertness scores. The statistical model contained fixed effect terms for Visit, Time, Treatment, Time-by-Treatment, Baseline-by-Treatment, and Subject as a random effect. Baseline VAS score was included as a covariate. The Baseline-by-Treatment term was not significant (p=0.6821) indicating that the mean baseline values across treatments were comparable.

The Baseline-by-Treatment term was dropped from the model and the analysis was rerun. The Time-by-treatment term was found to be nonsignificant (p=0.7048) indicating that the four treatments showed similar profiles over time.

When Time-by-Treatment was excluded from the model the Baseline and Time terms were highly statistically significant (p<0.0001), while the Treatment term had p=0.8573. The treatment means were all statistically comparable to each other. Within the ANCOVA, comparisons of the EC beverage to the IRC were performed using SAS Estimate statement for both the 160 mg dose strength (EC<IRC, difference=0.9424, p=0.5011) and the 250 mg dose strength (EC<IRC, difference=−0.7542, p=0.5877). The mean EC and IRC results, at both dose levels, were statistically comparable to each other. While the mean VAS for the 250 mg dose differed from that for the 160 mg dose (difference=0.2997), this was not detected as being statistically significant (p=0.8859). The mean results for the 250 mg dose were comparable to that for the 160 mg dose. Table 1 summarizes these results.

TABLE 1

| Repeated Measures | | |
|---|---|---|
| ANCOVA for | | |
| Alertness | p-value | Comment/Interpretation |
| Baseline | <0.0001 | Baseline is an appropriate covariate |
| Time | <0.0001 | Alertness at earlier post-dose times > than |
| | | at later ones |
| Treatment | 0.8573 | The 4 treatment means are statistically |
| | | equivalent to each other |
| Baseline*Treatment | 0.6821 | Baseline values across treatments are |
| | | statistically equivalent |
| Treatment*Time | 0.7048 | The 4 treatment profiles over time are |
| | | statistically comparable |
| 160 mg Dose: EC | 0.5011 | EC < IRC, Difference = 0.9424 |
| vs. IRC | | |
| 250 mg Dose: EC | 0.5877 | EC < IRC, Difference = 0.7542 |
| vs. IRC | | |
| 250 mg vs. 160 mg | 0.8859 | 250 mg > 160 mg, Difference = 0.2997 |
| Residual Standard | | 12.76 (Model with time and treatment |
| Deviation | | fixed effects) |

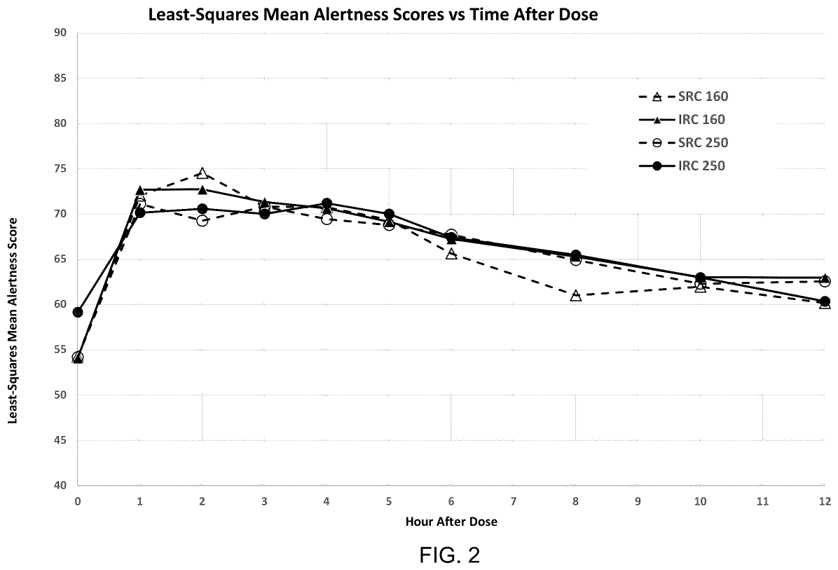

FIG. 2 illustrates the treatment profiles across time for Alertness.

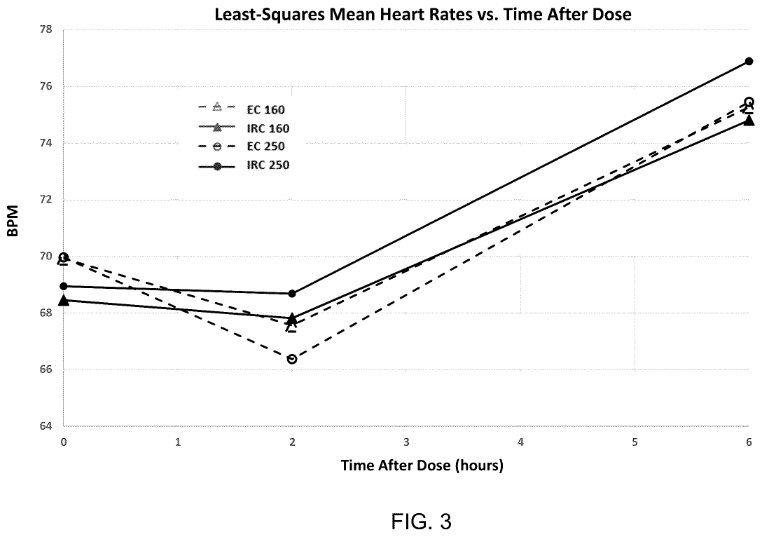

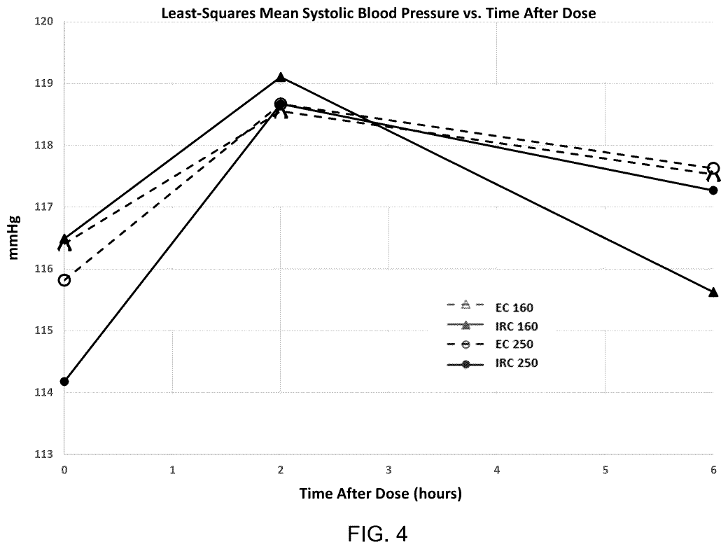

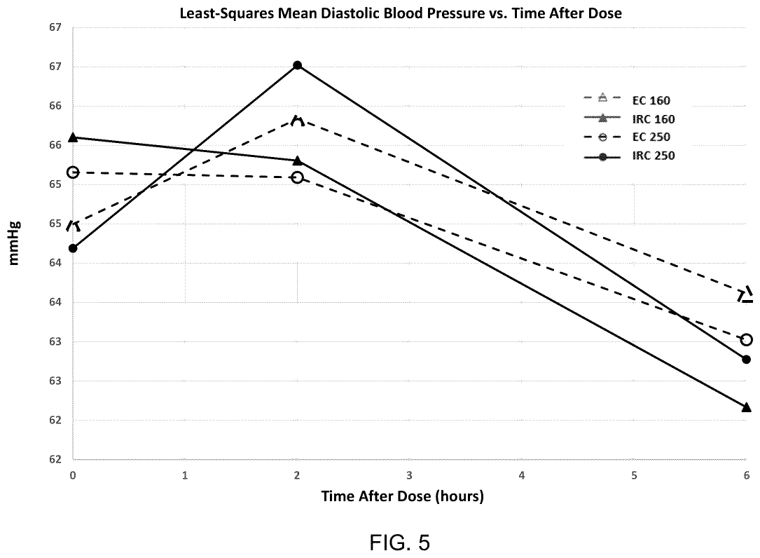

Statistical analyses of the vital signs heart rate, systolic blood pressure and diastolic blood pressure were conducted using SAS version 9.1.3. Proc GLM was used to evaluate the baseline results. The statistical model had terms for Subject and Treatment. The hypothesis of equal treatment means was not rejected at the 5% significance level for any of the three vital signs evaluated. The combined data for the 2-hour and 4-hour measurements, for each vital sign, were evaluated with Proc GLM. The statistical model contained terms for Subject, Treatment, Hour, and Treatment-by-Hour, with Baseline value included as a covariate. The Treatment-by-Hour term was not detected as statistically significant (i.e., p>0.05) for any of the three vital signs. Any difference between the 2-hour and 6-hour results was statistically consistent across the four treatments. The Treatment-by-Hour term was then excluded from the model. The 2-hour mean for each vital sign differed statistically (p<0.05) from that for the 6-hour mean. Tables 2-7 summarize the statistical results for the vital signs: heart rate (Tables 2 and 3); systolic blood pressure (Tables 4 and 5); and diastolic blood pressure (Tables 6 and 7). FIGS. 3-5 are graphs of the least-squares mean vital sign by time after dose for the four treatments.

TABLE 2

| | LSmean Heart Rate (bpm) | | | | | |
|---|---|---|---|---|---|---|
| | EC | IRC | EC | IRC | p-values | |
| Time | 160 | 160 | 250 | 250 | Trt*Hour | Trt |
| Baseline | 69.9 | 68.4 | 70.0 | 68.9 | — | 0.1554 |
| 2 | 67.6 | 67.8 | 66.4 | 68.7 | 0.6743 | 0.1502 |
| 6 | 75.3 | 74.8 | 75.4 | 76.9 | | |

bpm: beats per minute

TABLE 3

| | Treatment | |
|---|---|---|
| Time | LSmean | p-value |
| 2 | 67.6 | <0.0001 |
| 6 | 75.6 | |

TABLE 4

| | LSmean Systolic BP (mmHg) | | | | | |
|---|---|---|---|---|---|---|
| | EC | IRC | EC | IRC | p-values | |
| Time | 160 | 160 | 250 | 250 | Trt*Hour | Trt |
| Baseline | 116.4 | 116.5 | 115.8 | 114.2 | — | 0.0534 |
| 2 | 118.5 | 119.1 | 118.7 | 118.7 | 0.6092 | 0.8948 |
| 6 | 117.5 | 115.6 | 117.6 | 117.3 | | |

TABLE 5

| | Treatment | |
|---|---|---|
| Time | LSmean | p-value |
| 2 | 118.7 | 0.0213 |
| 6 | 117.0 | |

TABLE 6

| | LSmean Diastolic BP (mmHg) | | | | | |
|---|---|---|---|---|---|---|
| | EC | IRC | EC | IRC | p-values | |
| Time | 160 | 160 | 250 | 250 | Trt*Hour | Trt |
| Baseline | 64.5 | 65.6 | 65.2 | 64.2 | — | 0.1517 |
| 2 | 65.8 | 65.3 | 65.1 | 66.5 | 0.5287 | 0.3902 |
| 6 | 63.6 | 62.2 | 63.0 | 62.8 | | |

TABLE 7

| | Treatment | |
|---|---|---|
| Time | LSmean | p-value |
| 2 | 65.7 | <0.0001 |
| 6 | 62.9 | |

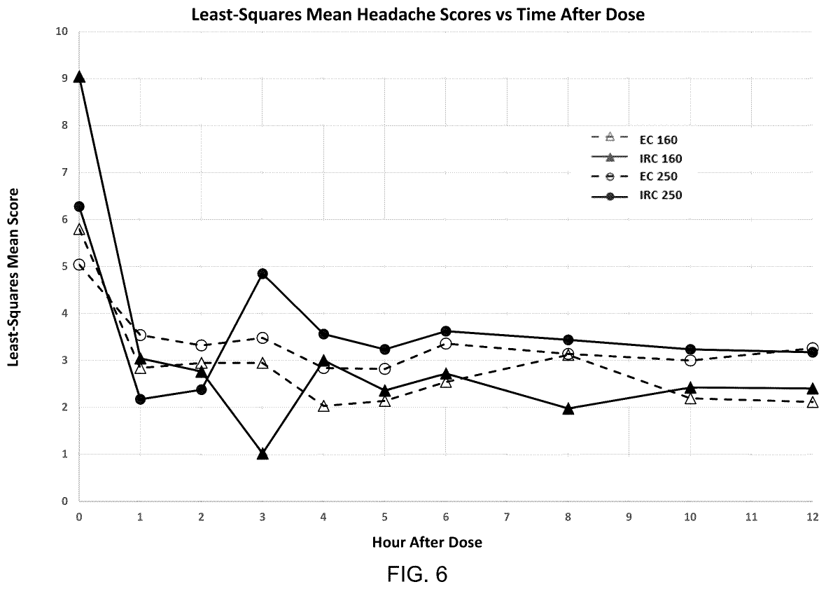

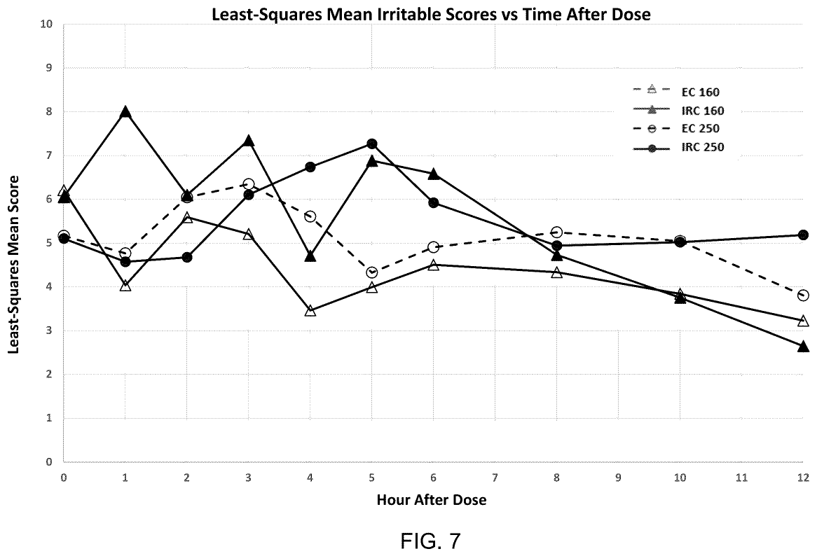

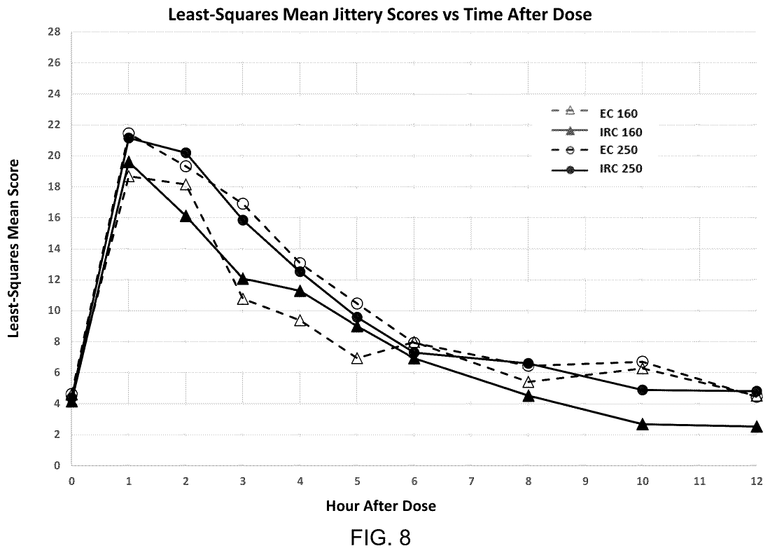

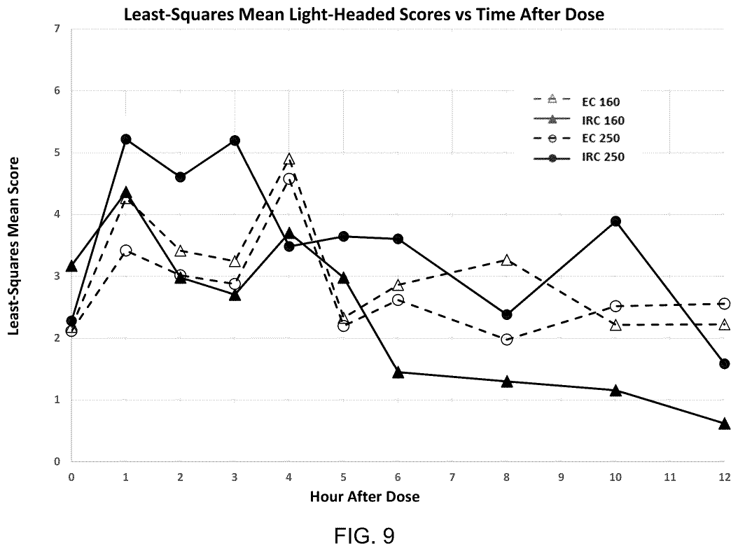

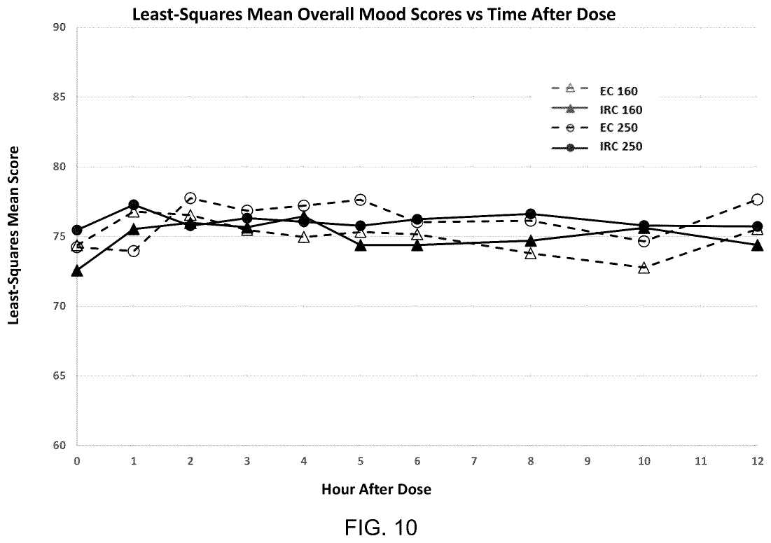

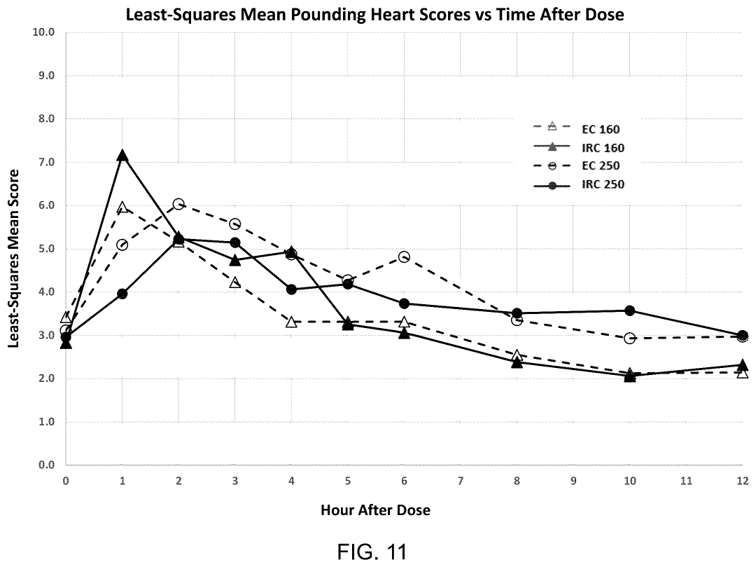

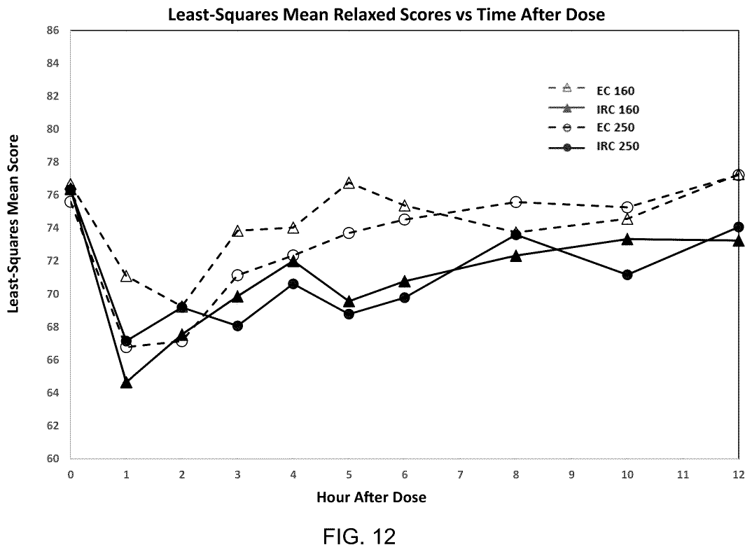

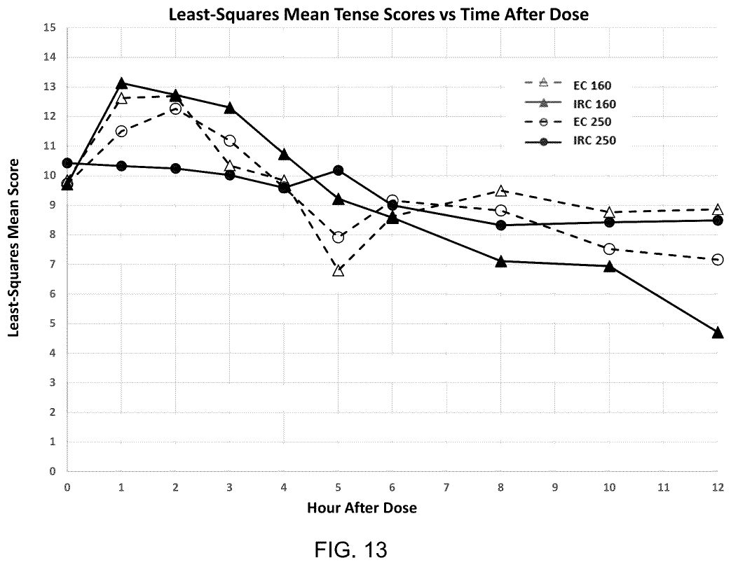

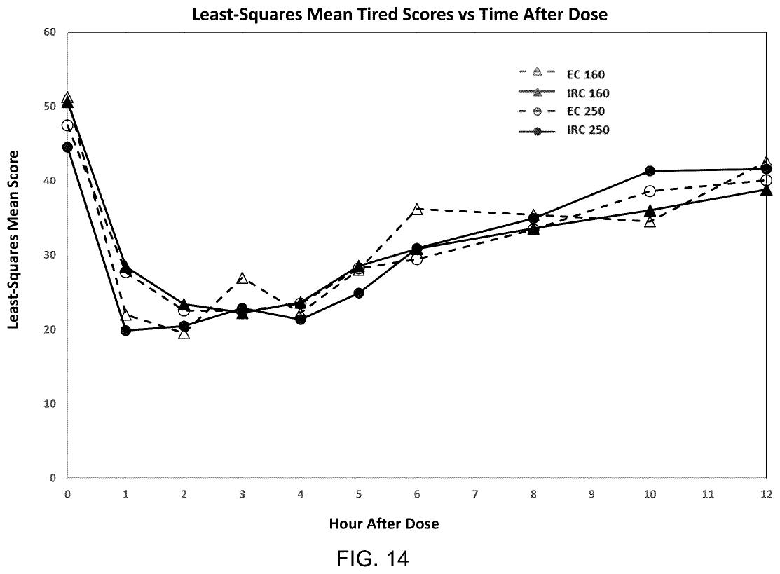

As no statistically significant (p<0.05) treatment difference was detected for the Alertness VAS results, according to the Statistical Analysis Plan, the other VAS parameters were not to be formally evaluated. For informational purposes, the results of the other VAS parameters were statistically evaluated. The statistical model contained terms for Time, Treatment and Treatment-by-Time interaction, with Baseline as the covariate. Residual standard deviation was obtained from a model that excluded all non-significant (p>0.05) terms except for Baseline and Time. Tables 8-16 and FIGS. 6-14 summarize these evaluations: headache (Table 8; FIG. 6); irritable (Table 9; FIG. 7); jittery (Table 10; FIG. 8); light-headed (Table 11; FIG. 9); overall mood (Table 12; FIG. 10); pounding heart (Table 13; FIG. 11); relaxed (Table 14; FIG. 12); tense (Table 15; FIG. 13); and tired (Table 16; FIG. 14).

TABLE 8

| Repeated Measures | | |
|---|---|---|
| ANCOVA for Headache | p-value | Value |
| Baseline | <0.0001 | |
| Time | <0.0001 | |
| Treatment | 0.7855 | |
| Treatment*Time | 0.4020 | |
| Residual Standard Deviation | | 6.98 |

FIG. 6 is a graph of the least-squares mean headache scores versus time after dose (hours).

TABLE 9

| Repeated Measures | | |
|---|---|---|
| ANCOVA for Irritable | p-value | Value |
| Baseline | <0.0001 | |
| Time | 0.0433 | |
| Treatment | 0.3286 | |
| Treatment*Time | 0.1766 | |
| Residual Standard Deviation | | 6.75 |

FIG. 7 is a graph of the least-squares mean irritable scores versus time after dose (hours).

TABLE 10

| Repeated Measures | | |
|---|---|---|
| ANCOVA for Jittery | p-value | Value |
| Baseline | <0.0001 | |
| Time | <0.0001 | |
| Treatment | 0.3323 | |
| Treatment*Time | 0.9881 | |
| Residual Standard Deviation | | 12.69 |

FIG. 8 is a graph of the least-squares mean jittery scores versus time after dose (hours).

TABLE 11

| Repeated Measures | | |
|---|---|---|
| ANCOVA for Light-Headed | p-value | Value |
| Baseline | <0.0001 | |
| Time | 0.0006 | |
| Treatment | 0.4215 | |
| Treatment*Time | 0.7257 | |
| Residual Standard Deviation | | 6.62 |

FIG. 9 is a graph of the least-squares mean light-headed scores versus time after dose (hours).

TABLE 12

| Repeated Measures | | |
|---|---|---|
| ANCOVA for Overall Mood | p-value | Value |
| Baseline | <0.0001 | |
| Time | 0.2765 | |
| Treatment | 0.4358 | |
| Treatment*Time | 0.8031 | |
| Residual Standard Deviation | | 9.85 |

FIG. 10 is a graph of the least-squares mean overall mood scores versus time after dose (hours).

TABLE 13

| Repeated Measures | | |
|---|---|---|
| ANCOVA for Pounding Heart | p-value | Value |
| Baseline | <0.0001 | |
| Time | <0.0001 | |
| Treatment | 0.5803 | |
| Treatment*Time | 0.6899 | |
| Residual Standard Deviation | | 5.34 |

FIG. 11 is a graph of the least-squares mean pounding heart scores versus time after dose (hours).

TABLE 14

| Repeated Measures | | |
|---|---|---|
| ANCOVA for Relaxed | p-value | Value |
| Baseline | <0.0001 | |
| Time | <0.0001 | |
| Treatment | 0.0331 | 160 mg: EC > IRC by 3.14 |
| Treatment*Time | 0.6896 | |
| Residual Standard Deviation | | 13.27 |

FIG. 12 is a graph of the least-squares mean relaxed scores versus time after dose (hours).

TABLE 15

| Repeated Measures | | |
|---|---|---|
| ANCOVA for Tense | p-value | Value |
| Baseline | <0.0001 | |
| Time | 0.0003 | |
| Treatment | 0.9646 | |
| Treatment*Time | 0.9703 | |
| Residual Standard Deviation | | 10.09 |

FIG. 13 is a graph of the least-squares mean tense scores versus time after dose (hours).

TABLE 16

| Repeated Measures | | |
|---|---|---|
| ANCOVA for Tired | p-value | Value |
| Baseline | <0.0001 | |
| Time | <0.0001 | |
| Treatment | 0.7638 | |
| Treatment*Time | 0.2274 | |
| Residual Standard Deviation | | 18.14 |

FIG. 14 is a graph of the least-squares mean tired scores versus time after dose (hours).

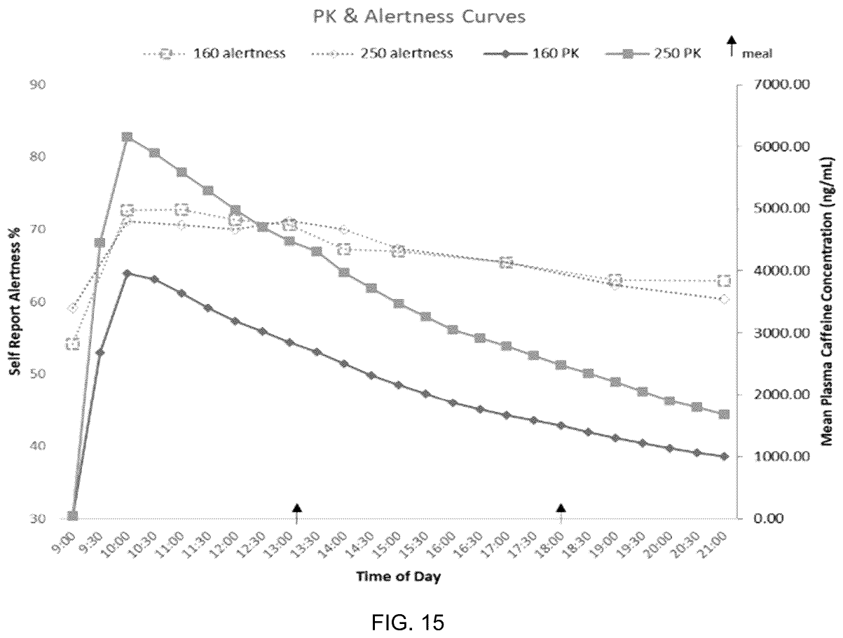

The mean plasma caffeine concentration (ng/mL) versus time of day was plotted alongside the self-reported alertness (%) versus time of day in FIG. 15. Both IRC samples (160 mg and 250 mg) showed similar peaks in the plasma concentration at about 1 hr post beverage consumption (the first sip being time zero). Once the maximum concentration was reached, the plasma concentrations trailed off over time. The half-life for both the 160 mg and 250 mg samples was about 5.5 hr. Meanwhile, the alertness levels similarly peaked at about 1 hr post beverage consumption but then the alertness level was statistically maintained over a 5 hr time period to provide sustained energy (e.g., consistent energy, long lasting energy). Meals were provided at the 4 hr and 9 hr post-ingestion time points (denoted by arrows on the x-axis).

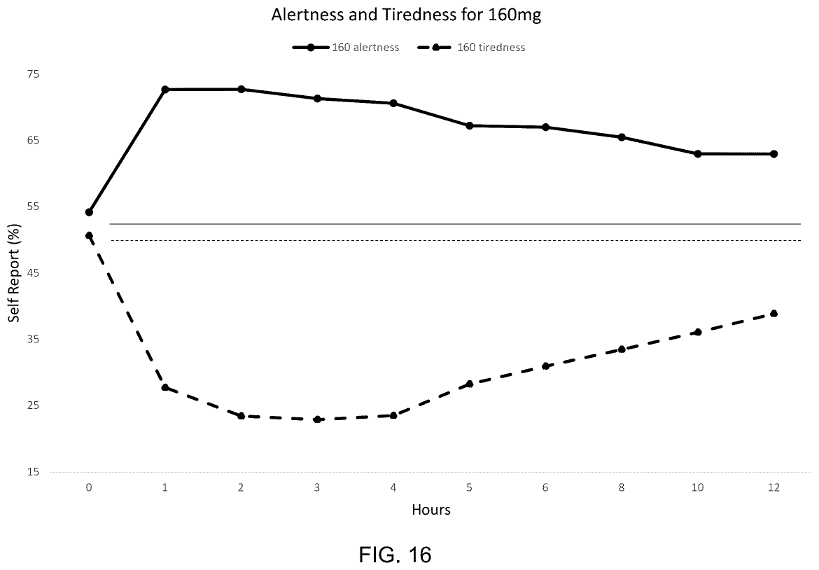

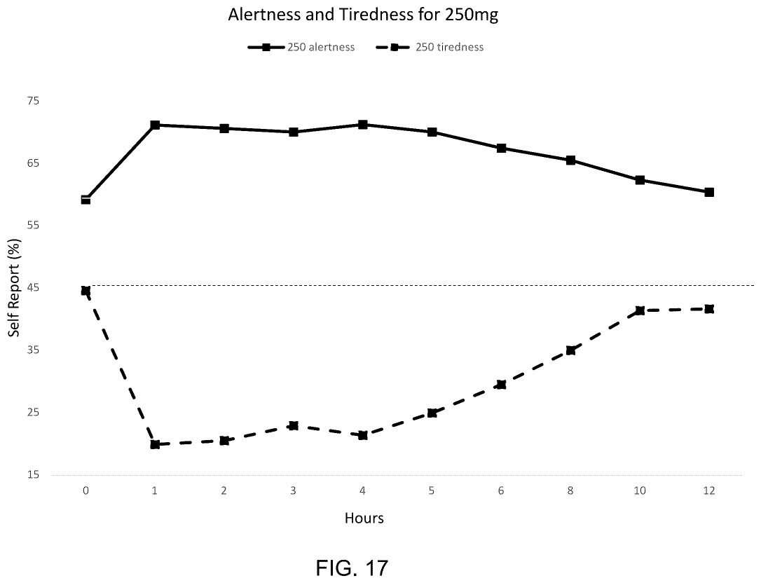

Alertness and tiredness are interconnected and inversely related. FIG. 16 illustrates alertness and tiredness VAS (%) versus hours post-consumption of a 160 mg IRC beverage. FIG. 17 illustrates alertness and tiredness VAS (%) versus hours post-consumption of a 250 mg caffeine beverage. When FIGS. 16 and 17 were compared to each other, it was observed that there was no statistical difference in alertness or tiredness at 160 mg or 250 mg from hour 1 (peak) to hour 5.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

1. A method of providing sustained energy from a beverage comprising one or more stimulants, the method comprising:
(a) administering the beverage comprising one or more stimulants to a consumer;
(b) measuring the consumer's plasma concentration of the one or more stimulants over a first time period;
(c) measuring the consumer's visual analog scale (VAS) response over a second time period; and
(d) increasing or decreasing the amount of the one or more stimulants in the beverage based on the measurements from (b) and (c).

2. A method of providing sustained energy from a beverage comprising one or more stimulants, the method comprising:
(a) administering the beverage comprising an amount of one or more stimulants to a consumer;
(b) measuring the consumer's plasma concentration of the amount of one or more stimulants over a first time period;
(c) measuring the consumer's visual analog scale (VAS) response over a second time period;
(d) generating, using a trained machine learning model, a stimulant amount based on the measurements from (b) and (c); and
(e) increasing or decreasing the amount of the one or more stimulants in the beverage to match the stimulant amount and provide an optimized beverage.

3. The method of claim 1, wherein the one or more stimulants is selected from the group consisting of a methylxanthine, a B vitamin, yerba mate, guarana, ephedra, taurine, carnitine, creatine, sucralose, maltodextrin, acai, glucuronolactone, yohimbine, ginseng, gingko biloba, kola nut, alpinia galanga, L-theanine, mango leaf, ornithine, blackcurrant, ashwagandha, coffeeberry, arginine silicate, curcumin, methylliberine, sideritis scardica, green oat extract, hawthorn, holy basil, lemon balm, Rhodiola rosea, rosemary, sage, spearmint, theacrine, tyrosine, cocoa flavanols, CoQ10, moringa, cacao, guayusa, and any combination thereof.

4.-5. (canceled)

6. The method of claim 1, wherein the one or more stimulants comprises caffeine.

7. The method of claim 1, wherein the beverage further comprises water and at least one additive selected from a sweetener, a preservative, color, a flavor, an antioxidant, a nutrient, and any combination thereof.

8. The method of claim 7, wherein the beverage further comprises water and at least one nutrient.

9. The method of claim 1, am wherein the first time period is about 30 minutes to about 15 hours after the beverage has been administered.

10. (canceled)

11. The method of claim 1, wherein the consumer's plasma concentration is measured about every 30 minutes during the first time period.

12. The method of claim 1, wherein the second time period is about 30 minutes to about 15 hours after the beverage has been administered.

13.-14. (canceled)

15. The method of claim 1, wherein the consumer's VAS response comprises measuring one or more factors selected from alertness, headache, irritability, jitteriness, lightheadedness, overall mood, relaxation, tension, tiredness, or any combination thereof.

16. The method of claim 1, further comprising measuring a baseline for each of the consumer's plasma concentration and VAS response prior to (a).

17. The method of claim 16, wherein the baseline is measured at about 5 to 60 minutes prior to (a).

18. The method of claim 1, further comprising measuring one or more vital signs of the consumer prior to (d) in a third time period.

19.-20. (canceled)

21. The method of claim 1, wherein the amount of the one or more stimulants in the beverage are increased or decreased to provide a beverage with sustained energy for about 2 hours or more.

22.-23. (canceled)

24. The method of claim 1, wherein the amount of the one or more stimulants in the beverage is increased based on a desired VAS response.

25. The method of claim 1, wherein the amount of the one or more stimulants in the beverage is decreased based on a desired VAS response.

26.-35. (canceled)

36. The method of claim 2, wherein the machine learning model is configured to determine the optimal value for the first time period or the second time period.

37. A method of providing sustained energy from a beverage comprising one or more stimulants, the method comprising:
a. providing user input to a formulation machine learning model, wherein the formulation machine learning model is trained to generate an optimized formulation based on the user input, and wherein the user input comprises a desired VAS response;
b. outputting, by the formulation machine learning model, the optimized formulation, wherein the optimized formulation comprises a combination of the one or more stimulants including at least one of an amount of the one or more stimulants and a type of the one or more stimulants; and
c. providing the optimized formulation to a beverage mixer for producing the beverage based on the combination of the one or more stimulants.

38. The method of claim 37, wherein the one or more stimulants is selected from the group consisting of a methylxanthine, a B vitamin, yerba mate, guarana, ephedra, taurine, carnitine, creatine, sucralose, maltodextrin, acai, glucuronolactone, yohimbine, ginseng, gingko biloba, kola nut, alpinia galanga, L-theanine, mango leaf, ornithine, blackcurrant, ashwagandha, coffeeberry, arginine silicate, curcumin, methylliberine, sideritis scardica, green oat extract, hawthorn, holy basil, lemon balm, Rhodiola rosea, rosemary, sage, spearmint, theacrine, tyrosine, cocoa flavanols, CoQ10, moringa, cacao, guayusa, and any combination thereof.

39.-41. (canceled)

42. The method of claim 37, wherein the beverage further comprises water and at least one additive selected from a sweetener, a preservative, color, a flavor, an antioxidant, a nutrient, and any combination thereof.

43.-48. (canceled)
